(19) United States
(12) Patent Application Publication
(10) Pub. No.: US 2026/0257997 A1
(43) Pub. Date: Sep. 3, 2026

(54) A METHOD FOR SYNTHESIZING ARYL ETHERS ORTHO-SUBSTITUTED TO THE NITROGEN ATOM OF N-HETEROCYCLES

(71) Applicant: Xinjiang University, Urumqi (CN)

(72) Inventors: Dong WANG, Urumqi (CN); Danyi LIU, Urumqi (CN); Fenlian XU, Urumqi (CN); Tong HAN, Urumqi (CN)

(21) Appl. No.: 19/444,759

(22) Filed: Jan. 9, 2026

(30) Foreign Application Priority Data
Feb. 24, 2025 (CN) 202510199195.5

(51) Int. Cl. C07D 217/24; C07D 213/64; C07D 215/227

(52) U.S. Cl. CPC C07D 217/24; C07D 213/64; C07D 215/227

(57) ABSTRACT

The present invention pertains to the technical field of synthesis and application of novel compounds, and discloses a synthesis method for aryl ethers substituted ortho to nitrogen in nitrogen-containing heterocycles. Under the catalysis of hexamethylphosphoramide (HMPA), using N,N-diisopropylethylamine (DIEA) as a base and with the assistance of POBr₃, the method employs nitrogen oxides to react with phenols, thereby achieving ortho-aryloxy substitution on nitrogen heterocycles. The method of the invention exhibits excellent atom economy (3a, 96%), green solvent selection (using ethyl acetate as solvent), mild reaction conditions, short reaction times (most substrates react within 5 minutes), broad substrate scope, and inexpensive and readily available starting materials. This method provides a convenient, efficient, green, and practical approach for synthesizing 1-aryloxyisoquinolines, 2-aryloxyquinolines, and 2-aryloxypyridine compounds.

### CROSS-REFERENCE TO RELATED APPLICATION

This application claims the benefit of priority to Chinese Application No. 202510199195.5, filed Feb. 24, 2025, now granted with publication number of CN 120058605 B, the contents of each of which are herein incorporated by reference in their entireties.

### TECHNICAL FIELD

The present invention relates to the field of synthesis and application technology of new compounds, especially a synthesis method for aryl ethers ortho-substituted to the nitrogen atom of N-heterocycles.

### BACKGROUND ART

Due to its unique chemical stability and functionality, aryl ethers have a wide range of applications in natural products, agrochemicals, bioactive compounds, drug synthesis, and optoelectronic materials, and are indispensable and important structural units in modern chemistry and materials science. For example, 1-aryloxyisoquinoline is widely used in the synthesis of pharmaceutical intermediates, including 6-bromo-1-phenoxyisoquinoline, 7-bromo-1-phenoxyisoquinoline, and 7-methyl-1-phenoxyisoquinoline. Compounds such as 1-phenoxyisoquinoline and 2-phenoxyquinoline exhibit certain antitumor activity. 1-aryloxyisoquinoline and 2-aryloxypyridine are also important intermediates in some optoelectronic materials.

Traditionally, aryl ethers ortho-substituted to the nitrogen in N-heterocycles are synthesized via transition-metal-catalyzed nucleophilic substitution between heterocyclic halides and phenols. However, this method typically requires harsh reaction conditions. Another method involves the use of easily accessible nitrogen-containing heterocyclic N-oxides as raw materials to undergo nucleophilic substitution reactions with phenols. However, this method requires the use of excessive activating agents and expensive or toxic reagents, which limits its application, and there is currently no economical, green and mild method for the synthesis of aryl ethers ortho-substituted to the nitrogen atom of N-heterocycles.

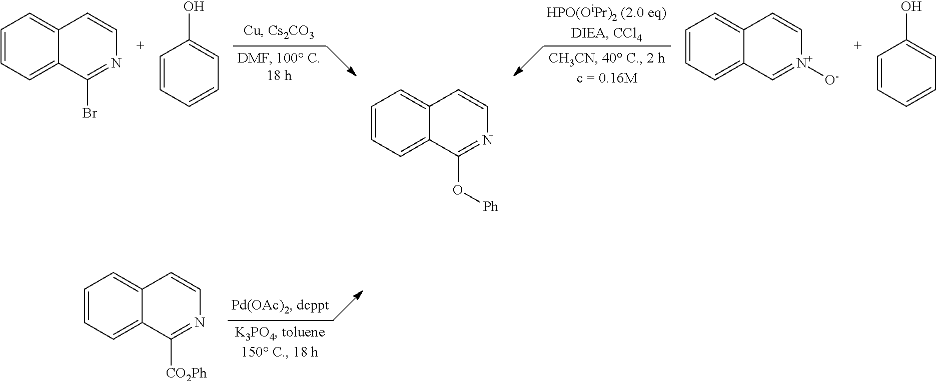

Based on the foregoing, there is a need for a practical, rapid, and environmentally benign synthetic method to access aryl ethers ortho-substituted to the nitrogen atom of N-heterocycles. The present invention provides a fast and green synthetic route to such ortho-substituted aryl ethers. In this method, N-heterocyclic N-oxides and phenol derivatives are used as substrates. With catalytic amounts of hexamethylphosphoramide (HMPA) and phosphoryl tribromide, an active phosphonium salt intermediate is generated in situ, which acts as an efficient activating agent to facilitate the reaction. This approach offers several advantages, including mild reaction conditions, in situ generation of the activator, and high catalytic efficiency.

### SUMMARY OF THE INVENTION

The purpose of the present invention is to overcome the shortcomings in the prior art and to provide a synthesis method for aryl ethers ortho-substituted to the nitrogen atom of N-heterocycles.

The technical scheme adopted by the present invention to solve the technical problem is:

A method for synthesizing an aryl ether ortho-substituted to the nitrogen atom of an N-heterocycle, characterized by: reacting an N-heterocyclic N-oxide with a phenol using HMPA as a catalyst, N,N-diisopropylethylamine (DIEA) as a base, and POBr₃ as an auxiliary agent, thereby effecting ortho-aryloxy substitution relative to the nitrogen atom of an N-heterocycle.

Further, the aryl ethers ortho-substituted to the nitrogen atom of N-heterocycles is an aryl heteroaryl ether, and the synthesis route is as follows:

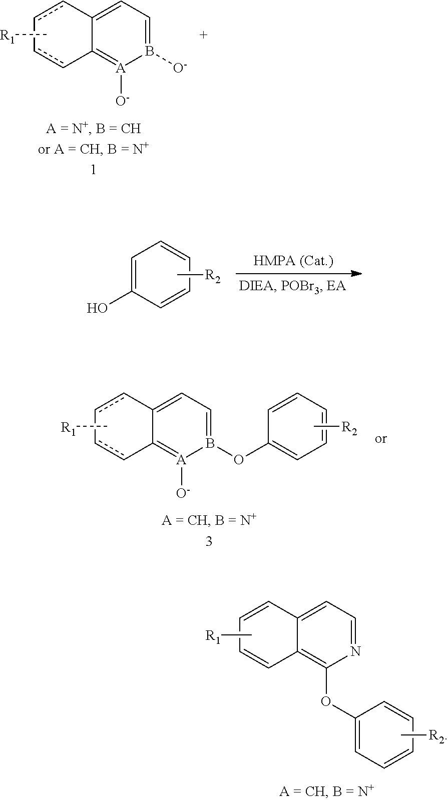

In some embodiments, in 1, A is N⁺ and B is CH, the synthetic route is as follows:

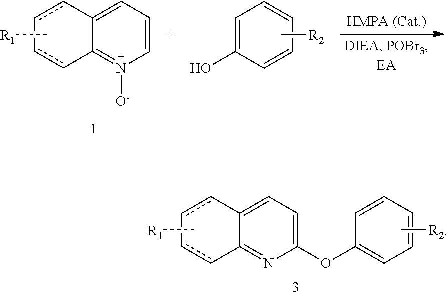

In some embodiments, in 1, A is CH and B is N⁺, the synthetic route is as follows:

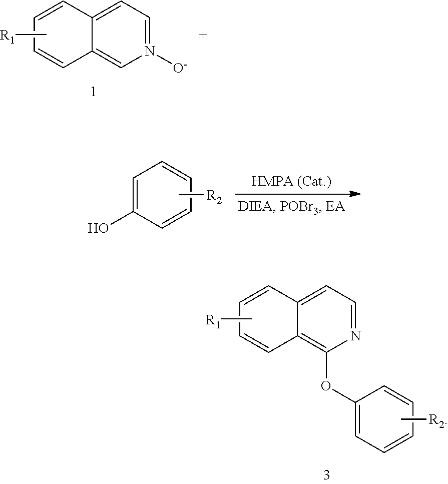

The N-heterocyclic N-oxide 1 is used as starting material, ethyl acetate (EA) is used as solvent, DIEA is used as the base, HMPA is used as the catalyst, and POBr₃ is used as the auxiliary agent, and compound 3 is obtained by reacting with phenol.

Among them, R₁ is selected from the group consisting of C1-C6 alkyl, halogen, C1-C6 ester, phenyl, and phenylethynyl; and R₂ is selected from the group consisting of C1-C6 alkyl, C1-C6 alkenyl, cyano, C1-C6 alkoxy, and halogen. Phenol substrates include pterostilbene.

Further, the aryl ether ortho-substituted to the nitrogen atom of N-heterocycles is 1-phenoxyisoquinoline, a compound with antitumor activity, and its synthesis route is as follows:

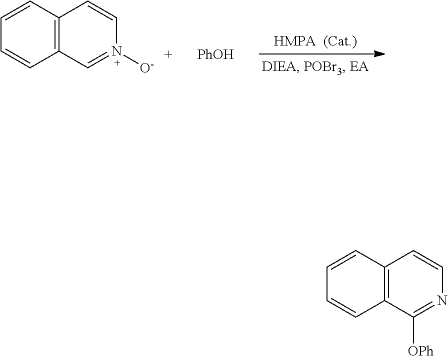

Isoquinoline-N-oxide is used as starting material, EA is used as solvent, DIEA is the base, HMPA is the catalyst, and POBr₃ is the auxiliary, and 1-phenoxyisoquinoline is obtained by reacting with phenol.

Further, the aryl ether ortho-substituted to the nitrogen atom of N-heterocycles is 6-bromo-1-phenoxyisoquinoline, a drug intermediate. The synthesis route is as follows:

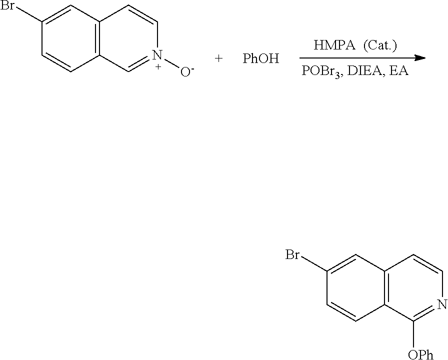

6-bromoisoquinoline 2-oxide is used as starting material, EA is used as solvent, DIEA is used as base, HMPA is used as catalyst, and POBr₃ is used as an auxiliary agent, and 6-bromo-1-phenoxyisoquinoline is obtained by reacting with phenol.

Further, the aryl ether ortho-substituted to the nitrogen atom of N-heterocycles is the drug intermediate 7-bromo-1-phenoxyisoquinoline, and the synthesis route is as follows:

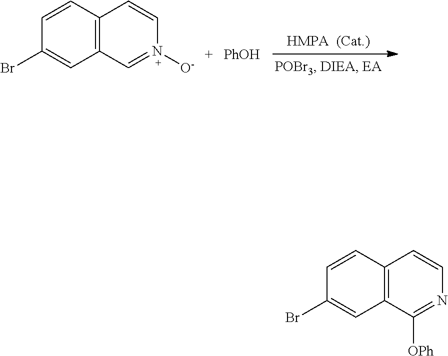

7-bromo-1-phenoxyisoquinoline is obtained by reacting 7-bromoisoquinoline nitrogen oxide with phenol, EA as solvent, DIEA as base, HMPA as catalyst and POBr₃ as auxiliary agent.

Further, the aryl ether ortho-substituted to the nitrogen atom of N-heterocycles is an intermediate of factor Xa inhibitor 7-methyl-1-phenoxyisoquinoline, and the synthesis route is as follows:

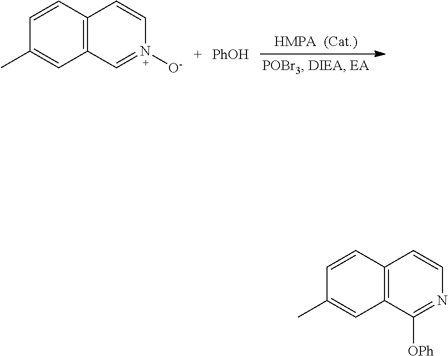

7-methylisoquinoline 2-oxide is used as starting material, EA is used as solvent, DIEA is the base, HMPA is used as the catalyst, and POBr₃ is used as the auxiliary, and 7-methyl-1-phenoxyisoquinoline is obtained by reacting with phenol.

Further, the aryl ether ortho-substituted to the nitrogen atom of N-heterocycles is a compound with antitumor activity 2-phenoxyquinoline, and the synthesis route is as follows:

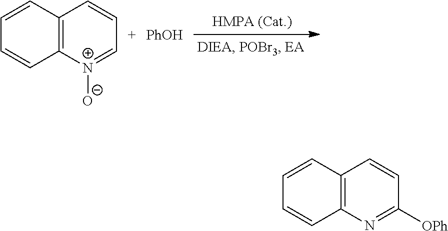 [SMILES: [O-][n+]1cccc2ccccc21 ; "PhOH" ; c1ccc(Oc2ccc3ccccc3n2)cc1]

Quinoline-N-oxide is used as starting material, EA is solvent, DIEA is the base, HMPA is the catalyst, and POBr₃ is the auxiliary, and 2-phenoxyquinoline is obtained by reacting with phenol.

Further, the aryl ether ortho-substituted to the nitrogen atom of N-heterocycles is a derivative of pterostilbene (E)-2-(4-(3,5-dimethoxystyrene) phenoxy) quinoline, and the synthesis route is as follows:

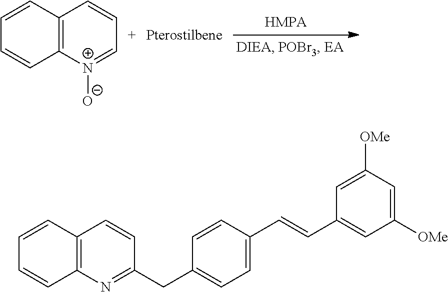

Using quinoline-N-oxide as starting material, EA as solvent, DIEA as base, HMPA as catalyst, and POBr₃ as auxiliary, (E)-2-(4-(3,5-dimethoxystyrene) phenoxy) quinoline) quinoline is obtained by reacting with pterostilbene.

Further, the aryl ether ortho-substituted to the nitrogen atom of N-heterocycles is 2-phenoxy-6-phenylpyridine, an intermediate of optoelectronic materials, and its synthesis route is as follows:

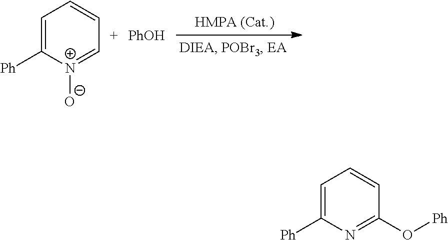 [SMILES: [O-][n+]1ccccc1-c1ccccc1 ; "PhOH" ; c1ccc(Oc2cccc(-c3ccccc3)n2)cc1]

2-phenoxy-6-phenylpyridine is obtained by reacting 2-phenylpyridine-1-oxide with phenol, EA as solvent, DIEA as base, HMPA as catalyst, and POBr₃ as auxiliary reagent.

Further, the aryl ether ortho-substituted to the nitrogen atom of N-heterocycles is selected from 1-aryloxyisoquinoline derivatives, 2-aryloxyquinoline derivatives and 2-aryloxypyridine derivatives;
wherein the structure of the 1-aryloxyisoquinoline derivative is selected from:

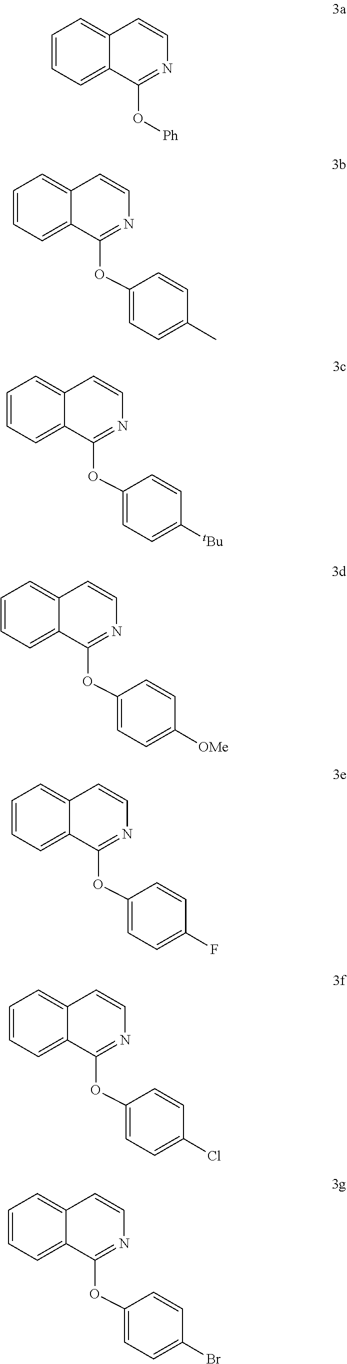

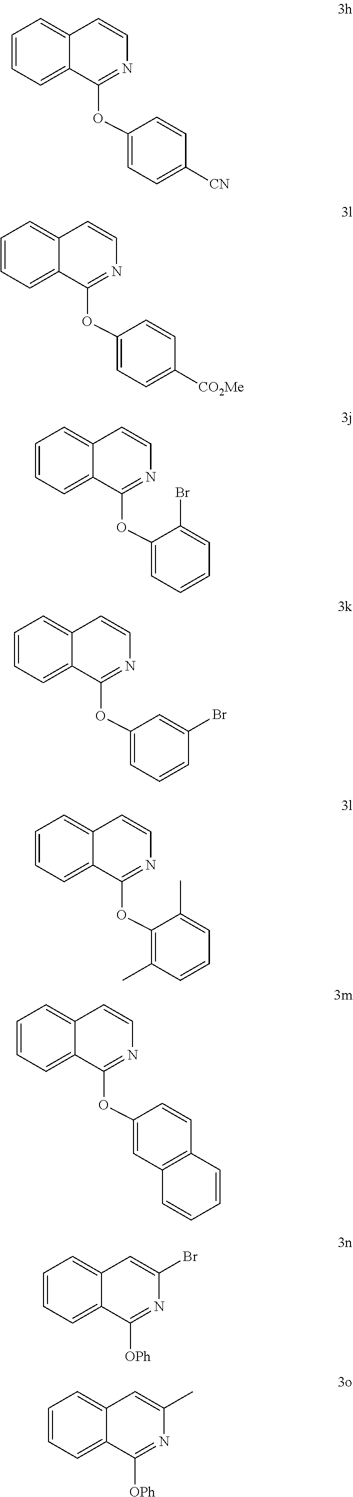

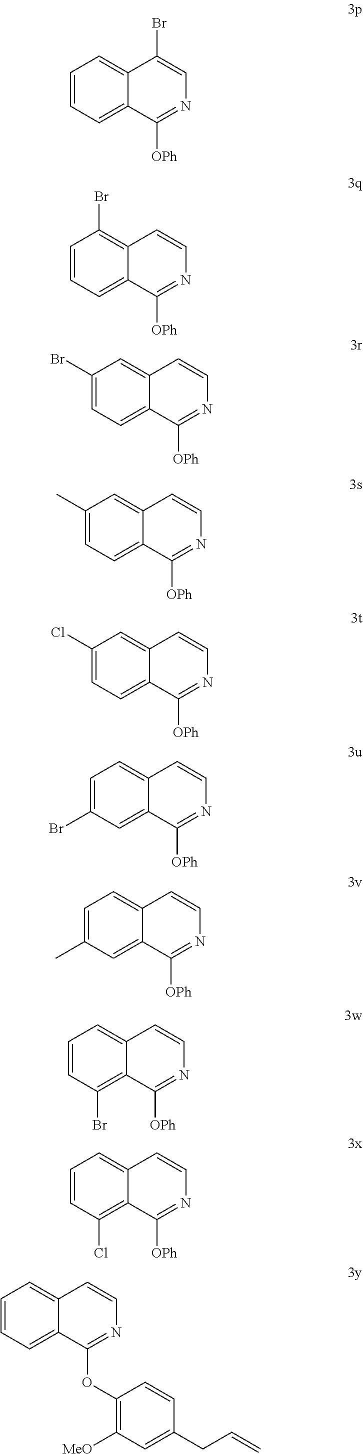

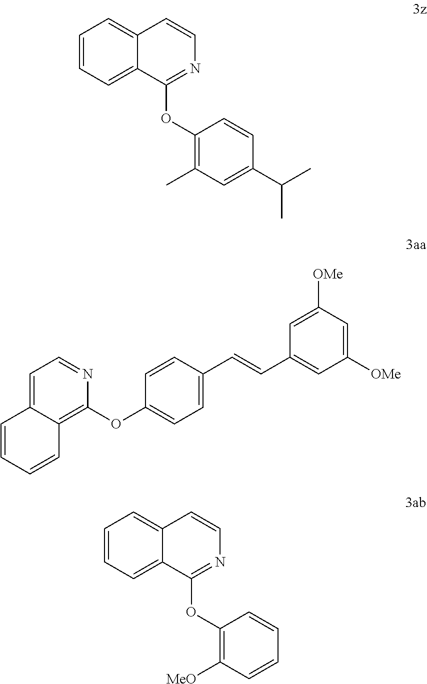 [SMILES: Cc1cc(C(C)C)ccc1Oc1nccc2ccccc12 ; COc1cc(/C=C/c2ccc(Oc3nccc4ccccc34)cc2)cc(OC)c1 ; COc1ccccc1Oc1nccc2ccccc12]

the structure of the 2-aryloxyquinoline derivative is selected from:

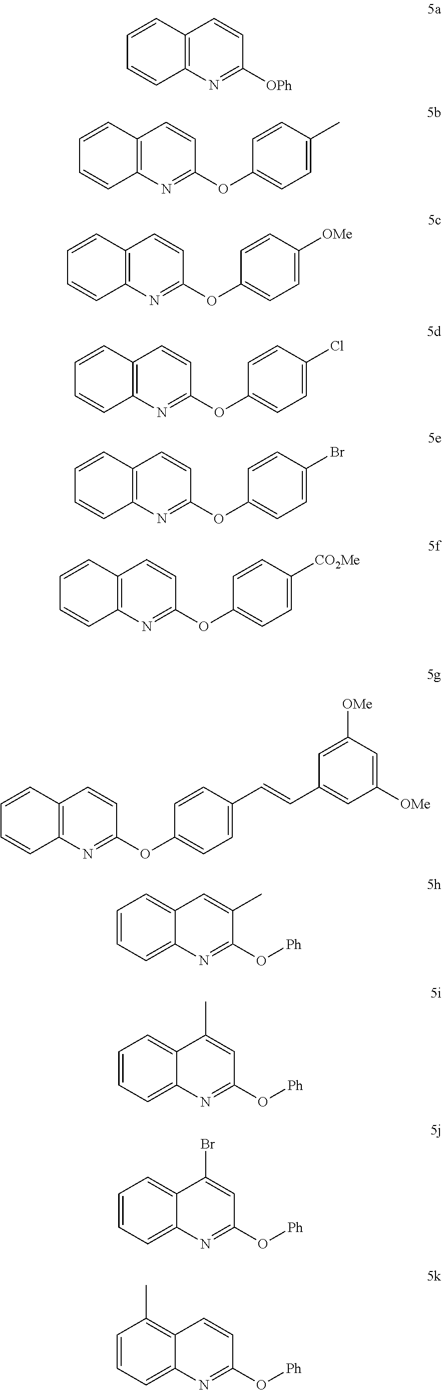

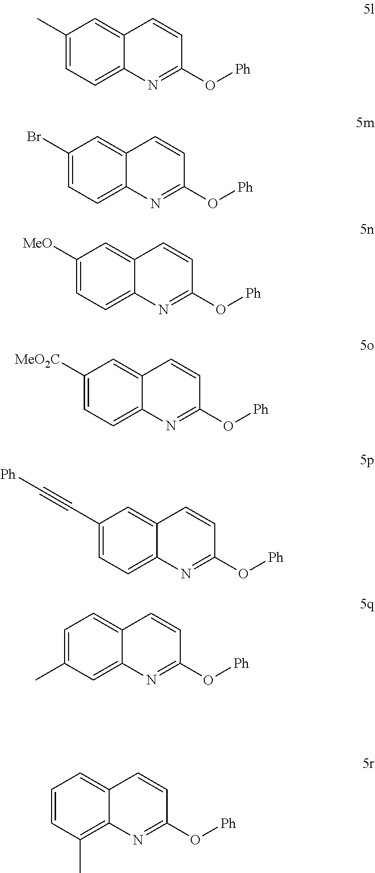

the structure of the 2-aryloxypyridine derivative is selected from:

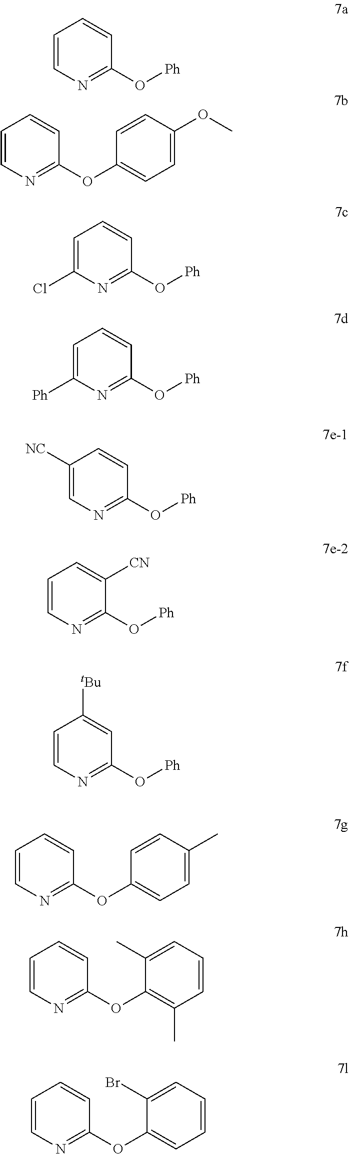

The advantages and positive effects of the present invention are:
1. The present invention provides a synthesis method for synthesizing aryl ethers ortho-substituted to the nitrogen atom of N-heterocycles, in which the method of the present invention involves: reacting an N-heterocyclic N-oxide with a phenol using HMPA as a catalyst, DIEA as a base, and POBr₃ as an auxiliary agent, thereby effecting ortho-aryloxy substitution relative to the nitrogen atom of an N-heterocycle. The method has the advantages of excellent atom economy (3a, 96%), green solvent (using ethyl acetate as solvent), mild reaction conditions, short reaction times (5 minutes for most substrates), wide range of substrates, and inexpensive, easily accessible substrates. This method provides a convenient, efficient, green and practical method for the synthesis of 1-aryloxyisoquinolines, 2-aryloxyquinolines and 2-aryloxypyridines.
2. The present invention provides a straightforward synthetic method for preparing aryl ethers ortho-substituted to the nitrogen atom of N-heterocycles. This approach offers advantages including operational simplicity, mild reaction conditions, and the use of low-cost, readily available substrates, making it economically valuable.
3. The present invention demonstrates high atom economy (e.g., 3a, 96%), requires no metal catalyst, allows recycling of by-products, and is environmentally benign.
4. The method features a broad substrate scope, successfully accommodating isoquinolines, quinolines, pyridines, and other analogues.
5. The strategy of in situ activating agent generation employed in this invention is unprecedented.
6. The invention utilizes inexpensive and readily available HMPA as the catalyst.
7. For the first time, this invention provides a general synthetic method for the 1-aryloxylation of isoquinoline derivatives.

### BRIEF DESCRIPTION OF THE DRAWINGS

FIG. 1 shows the nuclear magnetic hydrogen spectrum of compound 3c in deuterated chloroform in the present invention;

FIG. 2 shows the nuclear magnetic hydrogen spectrum of the compound 3g in deuterated chloroform in the present invention;

FIG. 3 shows the nuclear magnetic carbon spectrum of the compound 3g in deuterated chloroform in the present invention;

FIG. 4 shows the nuclear magnetic hydrogen spectrum of compound 3s in deuterated chloroform in the present invention;

FIG. 5 shows the nuclear magnetic carbon spectra of compound 3s in deuterated chloroform in the present invention;

FIG. 6 is the nuclear magnetic hydrogen spectrum of compound 5a in deuterated chloroform in the present invention.

### DETAILED DESCRIPTION OF EMBODIMENTS AND EXAMPLES

The present invention is further described below with reference to examples. These examples are illustrative rather than restrictive and should not be construed as limiting the scope of protection of the invention.

All experimental operations involved in the specific examples are conventional techniques in the field. For steps not specifically noted herein, those of ordinary skill in the art may carry them out with reference to common technical references, scientific literature, or related manuals and instructions available prior to the filing date of this application.

A method for synthesizing an aryl ether ortho-substituted to the nitrogen atom of an N-heterocycle, characterized by: reacting an N-heterocyclic N-oxide with a phenol using HMPA as a catalyst, DIEA as a base, and POBr₃ as an auxiliary agent, thereby effecting ortho-aryloxy substitution relative to the nitrogen atom of an N-heterocycle.

Preferably, the aryl ethers ortho-substituted to the nitrogen atom of N-heterocycles is an aryl heteroaryl ether. The synthetic route is shown below:

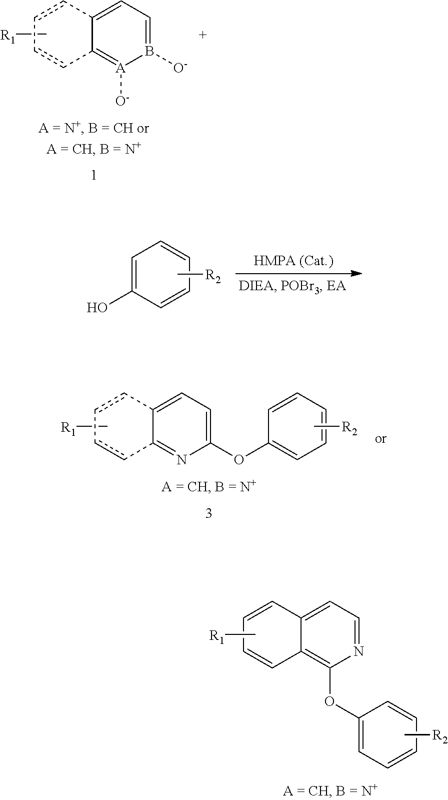

The N-heterocyclic N-oxide 1 is used as starting material, EA is used as solvent, DIEA is used as the base, HMPA is used as the catalyst, and POBr₃ is used as the auxiliary agent, and compound 3 is obtained by reacting with phenol.

Among them, R₁ is selected from the group consisting of C1-C6 alkyl, halogen, C1-C6 ester, phenyl, and phenylethynyl; and R₂ is selected from the group consisting of C1-C6 alkyl, C1-C6 alkenyl, cyano, C1-C6 alkoxy, and halogen. Phenol substrates include pterostilbene.

Preferably, the aryl ether ortho-substituted to the nitrogen atom of N-heterocycles is 1-phenoxyisoquinoline, a compound with antitumor activity. The synthetic route is shown below:

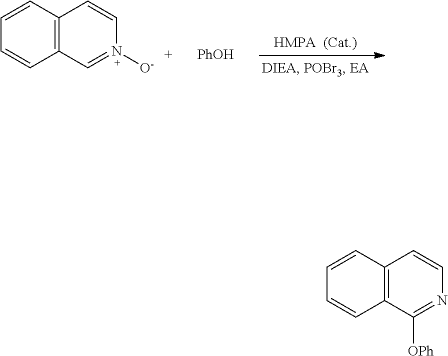

Isoquinoline-N-oxide is used as starting material, EA is solvent, DIEA is the base, HMPA is the catalyst, and POBr₃ is the auxiliary, and 1-phenoxyisoquinoline is obtained by reacting with phenol.

Preferably, the aryl ether ortho-substituted to the nitrogen atom of N-heterocycles is 6-bromo-1-phenoxyisoquinoline, a drug intermediate. The synthetic route is shown below:

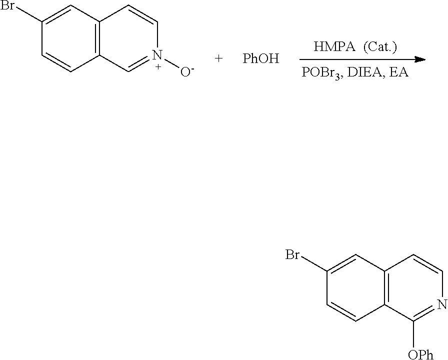

6-bromoisoquinoline 2-oxide is used as starting material, EA is used as solvent, DIEA is used as base, HMPA is used as catalyst, and POBr₃ is used as an auxiliary agent, and 6-bromo-1-phenoxyisoquinoline is obtained by reacting with phenol.

Preferably, the aryl ether ortho-substituted to the nitrogen atom of N-heterocycles is the drug intermediate 7-bromo-1-phenoxyisoquinoline. The synthetic route is shown below:

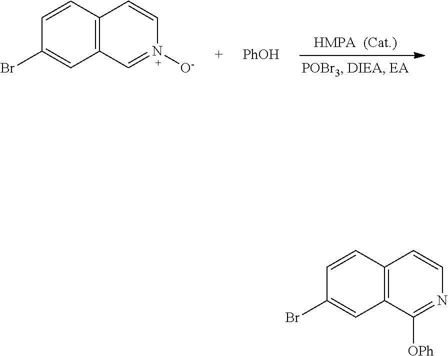

7-bromo-1-phenoxyisoquinoline is obtained by reacting 7-bromoisoquinoline nitrogen oxide with phenol, EA as solvent, DIEA as base, HMPA as catalyst and POBr₃ as auxiliary agent.

Preferably, the aryl ether ortho-substituted to the nitrogen atom of N-heterocycles is an intermediate of factor Xa inhibitor 7-methyl-1-phenoxyisoquinoline. The synthetic route is shown below:

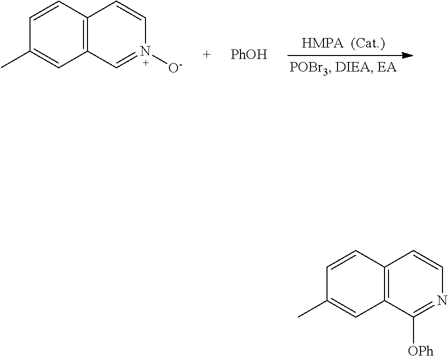

7-methylisoquinoline 2-oxide is used as starting material, EA is used as solvent, DIEA is the base, HMPA is used as the catalyst, and POBr₃ is used as the auxiliary, and 7-methyl-1-phenoxyisoquinoline is obtained by reacting with phenol.

Preferably, the aryl ether ortho-substituted to the nitrogen atom of N-heterocycles is a compound with antitumor activity 2-phenoxyquinoline. The synthetic route is shown below:

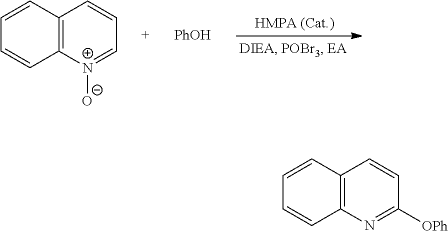 [SMILES: [O-][n+]1cccc2ccccc21 ; "PhOH" ; c1ccc(Oc2ccc3ccccc3n2)cc1]

Quinoline-N-oxide is used as raw material, EA is solvent, DIEA is the base, HMPA is the catalyst, and POBr₃ is the auxiliary, and 2-phenoxyquinoline is obtained by reacting with phenol.

Preferably, the aryl ether ortho-substituted to the nitrogen atom of N-heterocycles is a derivative of pterostilbene (E)-2-(4-(3,5-dimethoxystyrene) phenoxy) quinoline. The synthetic route is shown below:

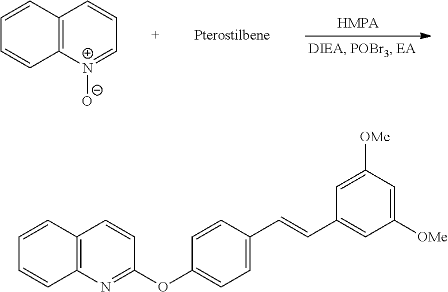 [SMILES: [O-][n+]1cccc2ccccc21 ; "Pterostilbene" ; COc1cc(/C=C/c2ccc(Oc3ccc4ccccc4n3)cc2)cc(OC)c1]

Using quinoline-N-oxide as starting material, EA as solvent, DIEA as base, HMPA as catalyst, and POBr₃ as auxiliary, (E)-2-(4-(3,5-dimethoxystyrene) phenoxy) quinoline) quinoline is obtained by reacting with pterostilbene.

Preferably, the aryl ether ortho-substituted to the nitrogen atom of N-heterocycles is 2-phenoxy-6-phenylpyridine, an intermediate of optoelectronic materials. The synthetic route is shown below:

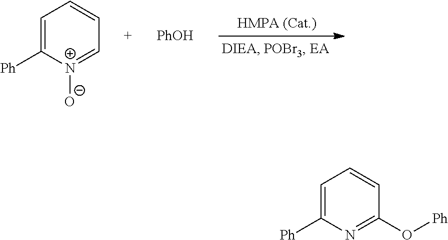 [SMILES: [O-][n+]1ccccc1-c1ccccc1 ; "PhOH" ; c1ccc(Oc2cccc(-c3ccccc3)n2)cc1]

2-phenoxy-6-phenylpyridine is obtained by reacting 2-phenylpyridine-1-oxide with phenol, EA as solvent, DIEA as base, HMPA as catalyst, and POBr₃ as auxiliary reagent.

Preferably, the aryl ether ortho-substituted to the nitrogen atom of N-heterocycles is selected from 1-aryloxyisoquinoline derivatives, 2-aryloxyquinoline derivatives and 2-aryloxypyridine derivatives;
wherein the structure of the 1-aryloxyisoquinoline derivative is selected from:

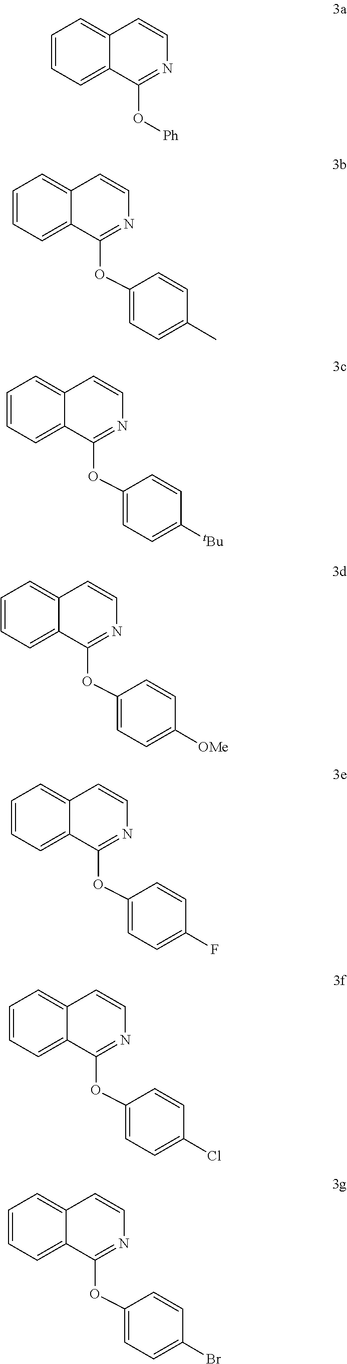

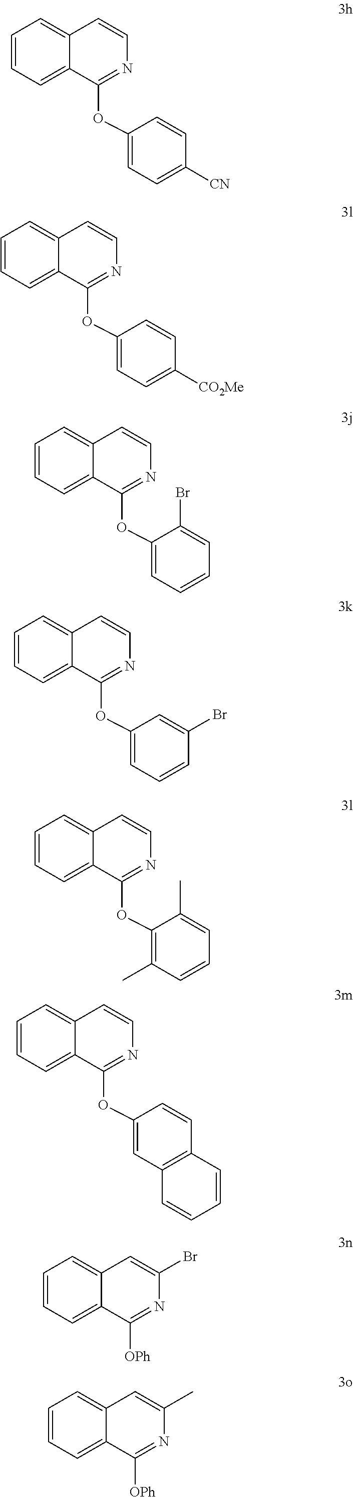

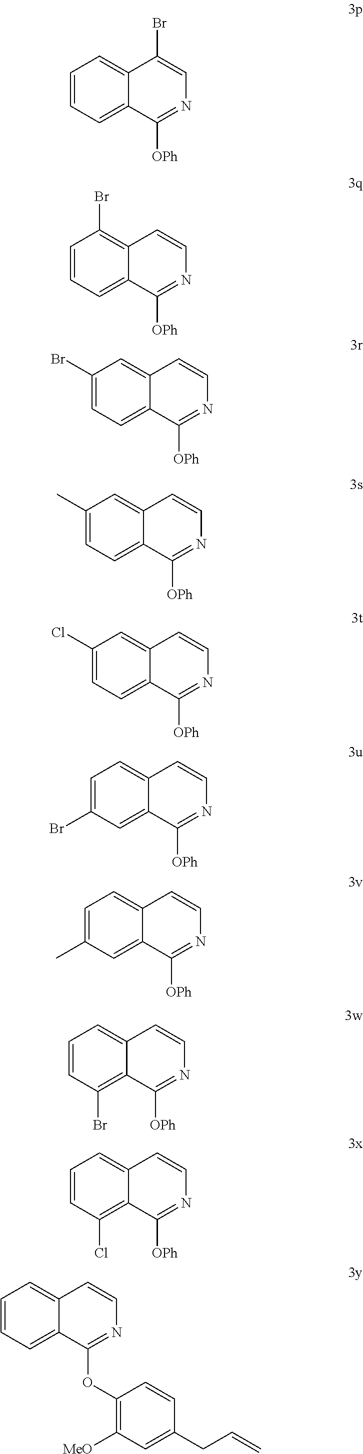

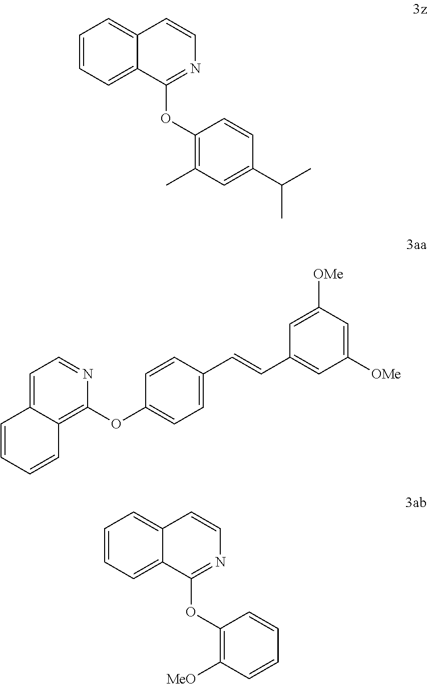 [SMILES: Cc1cc(C(C)C)ccc1Oc1nccc2ccccc12 ; COc1cc(/C=C/c2ccc(Oc3nccc4ccccc34)cc2)cc(OC)c1 ; COc1ccccc1Oc1nccc2ccccc12]

the structure of the 2-aryloxyquinoline derivative is selected from:

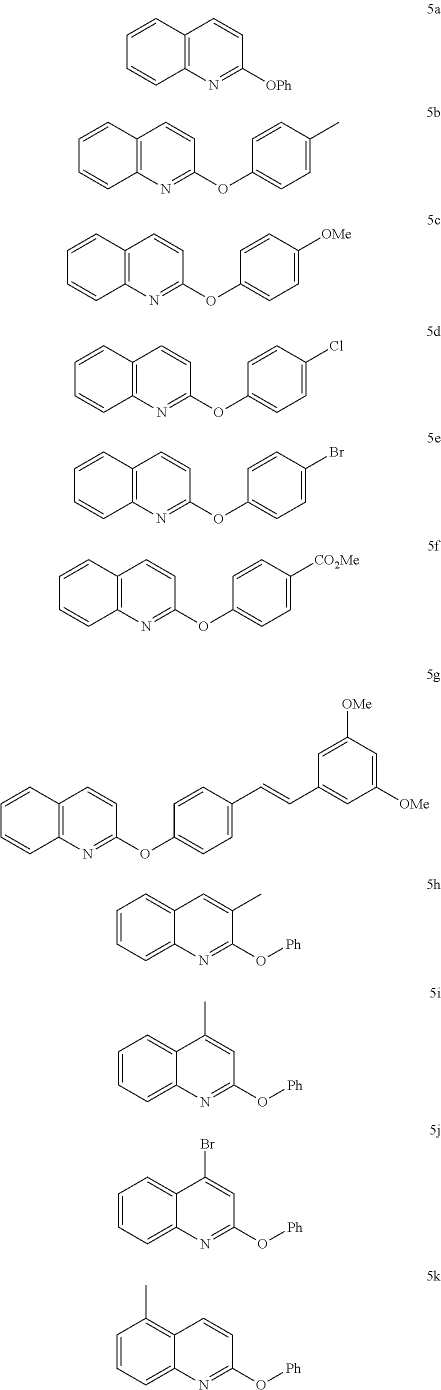

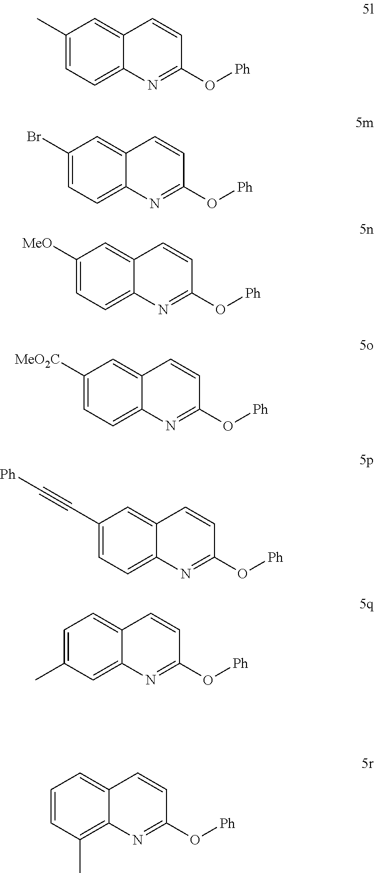

the structure of the 2-aryloxypyridine derivative is selected from:

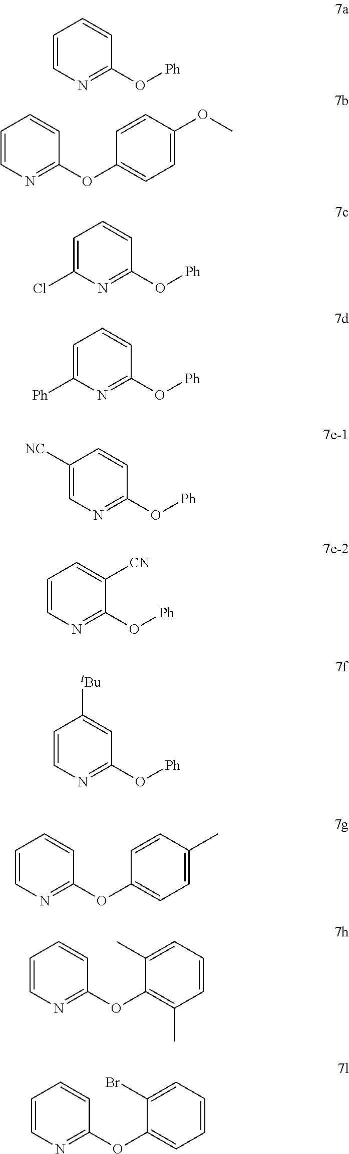

In some embodiments, the method herein is for synthesizing 1-aryloxyisoquinoline. In some embodiments, the 1-aryloxyisoquinoline can be any of those corresponding specific Examples described herein below. A general procedure according to Method 1 herein can be typically used for the synthesis of 1-aryloxyisoquinoline.

In some embodiments, the method herein is for synthesizing 2-aryloxyquinoline. In some embodiments, the 2-aryloxyquinoline can be any of those corresponding Examples described herein below. A general procedure according to Method 2 herein can be typically used for the synthesis of 2-aryloxyquinoline.

In some embodiments, the method herein is for synthesizing 2-aryloxypyridine. In some embodiments, the 2-aryloxypyridine can be any of those corresponding specific Examples described herein below. A general procedure according to Method 3 herein can be typically used for the synthesis of 2-aryloxypyridine.

Specifically, the relevant preparation and testing are as follows:

Method 1: A green synthesis method for 1-aryloxyisoquinoline, the steps are as follows: To a solution of an isoquinoline N-oxide derivative (1.0 mmol) in dry EA (1.0 mL) is added ArOH (3.0 mmol), DIEA (2.0 mmol) and HMPA (0.4 mmol). The resulting solution is cooled to 0° C., followed by the dropwise addition of a solution of POBr₃ (2.0 mmol) in EA (1.0 mL). The reaction mixture is then warmed to room temperature and stirred for 5 minutes. The reaction is quenched with a saturated potassium carbonate solution and the organic phase is separated. The aqueous phase is extracted once with EA. The organic phase is combined, dried over Na₂SO₄ and concentrated in vacuo to give the crude product. Purification by flash column chromatography (PE:EA=200:1-100:1) furnishes the desired product. PE refers to petroleum ether.

The present invention is further illustrated in detail by the following embodiments.

### Example 1

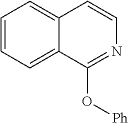 [SMILES: c1ccc(Oc2nccc3ccccc23)cc1]

To a solution of an isoquinoline N-oxide derivative (100 mg, 0.69 mmol) in EA (0.7 mL) was added phenol (198 mg, 2.1 mmol), DIEA (181 mg, 1.4 mmol) and HMPA (50 mg, 0.28 mmol). The resulting solution was cooled to 0° C., followed by the dropwise addition of a solution of POBr₃ (401 mg, 1.4 mmol) in EA (0.7 mL). The reaction mixture was then warmed to room temperature and stirred for 5 minutes. The reaction was quenched with a saturated potassium carbonate solution and the organic phase was separated. The aqueous phase was extracted once with EA. The organic phase was combined, dried over Na₂SO₄ and concentrated in vacuo to give the crude product. Purification by flash column chromatography (PE:EA=200:1-100:1) furnished the desired product (110 mg, 72% yield). Structural parameters: ¹H NMR (600 MHZ, CDCl₃) δ 8.35 (d, J=7.8 Hz, 1H), 7.88 (d, J=5.4 Hz, 1H), 7.71-7.70 (m, 1H), 7.63 (t, J=7.8 Hz, 1H), 7.53 (t, J=7.8 Hz, 1H), 7.36 (t, J=7.8 Hz, 2H), 7.22 (d, J=6.0 Hz, 1H), 7.18-7.15 (m, 3H).

### Example 2

To a solution of an isoquinoline N-oxide derivative (1.0 g, 6.9 mmol) in EA (7 mL) was added phenol (1.95 g, 20.7 mmol), DIEA (1.78 g, 13.8 mmol) and HMPA (0.50 g, 2.8 mmol). The resulting solution was cooled to 0° C., followed by the dropwise addition of a solution of POBr₃ (3.96 g, 13.8 mmol) in EA (7 mL). The reaction mixture was then warmed to room temperature and stirred for 5 minutes. The reaction was quenched with a saturated potassium carbonate solution and the organic phase was separated. The aqueous phase was extracted once with EA. The organic phase was combined, dried over Na₂SO₄ and concentrated in vacuo to give the crude product. Purification by flash column chromatography (PE:EA=200:1-100:1) furnished the desired product (967 mg, 63% yield). Structural parameters: ¹H NMR (600 MHZ, CDCl₃) δ 8.35 (d, J=7.8 Hz, 1H), 7.88 (d, J=5.4 Hz, 1H), 7.71-7.70 (m, 1H), 7.63 (t, J=7.8 Hz, 1H), 7.53 (t, J=7.8 Hz, 1H), 7.36 (t, J=7.8 Hz, 2H), 7.22 (d, J=6.0 Hz, 1H), 7.18-7.15 (m, 3H).

### Example 3

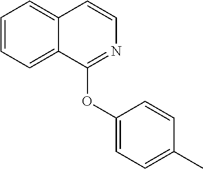 [SMILES: Cc1ccc(Oc2nccc3ccccc23)cc1]

The synthesis method of Example 3 is the same as the general method 1 above.

Reaction yield: 65%; Structural parameters: ¹H NMR (600 MHZ, CDCl₃) δ 8.44 (d, J=8.4 Hz, 1H), 7.96 (d, J=6.0 Hz, 1H), 7.78 (d, J=8.4 Hz, 1H), 7.72-7.69 (m, 1H), 7.62-7.59 (m, 1H), 7.28 (d, J=5.4 Hz, 1H), 7.25-7.24 (m, 2H), 7.14-7.13 (m, 2H), 2.38 (s, 3H).

### Example 4

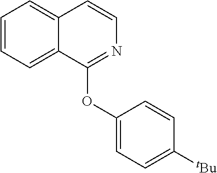 [SMILES: CC(C)(C)c1ccc(Oc2nccc3ccccc23)cc1]

The synthesis method of Example 4 is the same as the general method 1 above.

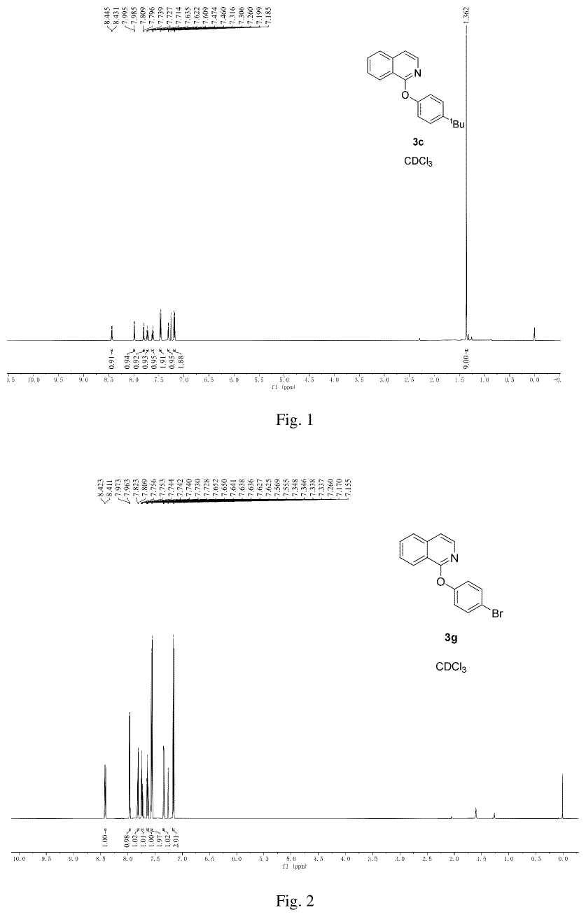

Reaction yield: 43%; Structural parameters: ¹H NMR (600 MHZ, CDCl₃) δ 8.44 (d, J=8.4 Hz, 1H), 7.99 (d, J=6.0 Hz, 1H), 7.80 (d, J=7.8 Hz, 1H), 7.73 (t, J=7.8 Hz, 1H), 7.62 (t, J=7.8 Hz, 1H), 7.47-7.46 (m, 2H), 7.31 (d, J=6.0 Hz, 1H), 7.20-7.19 (m, 2H), 1.36 (s, 9H). ¹H NMR spectra is shown in FIG. 1.

### Example 5

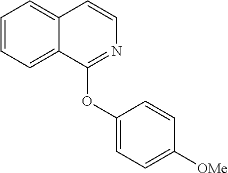 [SMILES: COc1ccc(Oc2nccc3ccccc23)cc1]

The synthesis method of Example 5 is the same as the general method 1 above.

Reaction yield: 52%; Structural parameters: ¹H NMR (600 MHZ, CDCl₃) δ 8.45 (d, J=9.6 Hz, 1H), 7.97 (d, J=6.0 Hz, 1H), 7.80-7.79 (m, 1H), 7.74-7.71 (m, 1H), 7.64-7.61 (m, 1H), 7.30 (d, J=6.6 Hz, 1H), 7.20-7.18 (m, 2H), 7.00-6.98 (m, 2H), 3.84 (s, 3H).

### Example 6

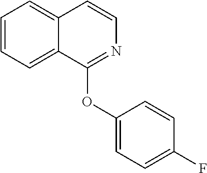 [SMILES: Fc1ccc(Oc2nccc3ccccc23)cc1]

The synthesis method of Example 6 is the same as the above general method 1.

Reaction yield: 67%; Structural parameters: ¹H NMR (600 MHZ, CDCl₃) δ 8.44 (d, J=8.4 Hz, 1H), 7.97 (d, J=5.4 Hz, 1H), 7.82-7.81 (d, J=7.8 Hz, 1H), 7.76-7.73 (m, 1H), 7.66-7.63 (m, 1H), 7.33 (d, J=5.4 Hz, 1H), 7.25-7.22 (m, 2H), 7.16-7.13 (m, 2H).

### Example 7

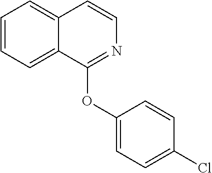 [SMILES: Clc1ccc(Oc2nccc3ccccc23)cc1]

The synthesis method of Example 7 is the same as the general method 1 above.

Reaction yield: 70%; Structural parameters: ¹H NMR (600 MHz, CDCl₃) δ 8.42 (d, J=9.6 Hz, 1H), 7.97 (d, J=6.0 Hz, 1H), 7.80 (d, J=7.8 Hz, 1H), 7.76-7.73 (m, 1H), 7.65-7.63 (m, 1H), 7.42-7.41 (m, 2H), 7.34 (d, J=6.6 Hz, 1H), 7.22-7.21 (m, 2H).

### Example 8

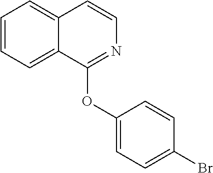 [SMILES: Brc1ccc(Oc2nccc3ccccc23)cc1]

The synthesis method of Example 8 is the same as the general method 1 above.

Reaction yield: 45%; Structural parameters: ¹H NMR (600 MHZ, CDCl₃) δ 8.42 (d, J=9.0 Hz, 1H), 7.97 (d, J=6.0 Hz, 1H), 7.82-7.81 (m, 1H), 7.76-7.73 (m, 1H), 7.65-7.63 (m, 1H), 7.57-7.56 (m, 2H), 7.34 (d, J=6.6 Hz, 1H), 7.17-7.16 (m, 2H). The ¹H NMR spectrum is shown in FIG. 2, and the 13C NMR spectrum is shown in FIG. 3.

### Example 9

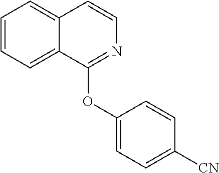 [SMILES: N#Cc1ccc(Oc2nccc3ccccc23)cc1]

The synthesis method of Example 9 is the same as the general method 1 above.

Reaction yield: 45%; Structural parameters: ¹H NMR (600 MHZ, CDCl₃) δ 8.38 (d, J=8.4 Hz, 1H), 7.98 (d, J=6.0 Hz, 1H), 7.85-7.84 (m, 1H), 7.78-7.75 (m, 1H), 7.74-7.73 (m, 2H), 7.67-7.65 (m, 1H), 7.41 (d, J=6.0 Hz, 1H), 7.39-7.38 (m, 2H).

### Example 10

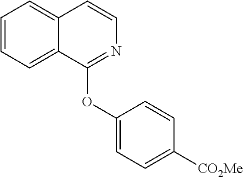 [SMILES: COC(=O)c1ccc(Oc2nccc3ccccc23)cc1]

The synthesis method of Example 10 is the same as the general method 1 above.

Reaction yield: 40%; Structural parameters: ¹H NMR (600 MHZ, CDCl₃) δ 8.41 (d, J=7.8 Hz, 1H), 8.15-8.14 (m, 2H), 7.99 (d, J=6.0 Hz, 1H), 7.84-7.82 (m, 1H), 7.76-7.74 (m, 1H), 7.66-7.63 (m, 1H), 7.38 (d, J=5.4 Hz, 1H), 7.34-7.32 (m, 2H), 3.93 (s, 3H).

### Example 11

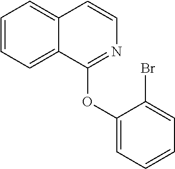 [SMILES: Brc1ccccc1Oc1nccc2ccccc12]

The synthesis method of Example 11 is the same as the general method 1 above.

Reaction yield: 46%; Structural parameters: ¹H NMR (600 MHZ, CDCl₃) δ 8.51 (d, J=7.8 Hz, 1H), 7.95 (d, J=5.4 Hz, 1H), 7.83-7.82 (m, 1H), 7.77-7.74 (m, 1H), 7.70-7.66 (m, 2H), 7.44-7.41 (m, 1H), 7.36-7.34 (m, 2H), 7.18-7.15 (m, 1H).

### Example 12

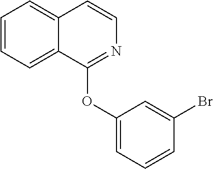 [SMILES: Brc1cccc(Oc2nccc3ccccc23)c1]

The synthesis method of Example 12 is the same as the general method 1 above.

Reaction yield: 43%; Structural parameters: ¹H NMR (600 MHZ, CDCl₃) δ 8.51 (d, J=7.8 Hz, 1H), 7.95 (d, J=5.4 Hz, 1H), 7.83-7.82 (m, 1H), 7.77-7.74 (m, 1H), 7.70-7.66 (m, 2H), 7.44-7.41 (m, 1H), 7.36-7.34 (m, 2H), 7.18-7.15 (m, 1H).

### Example 13

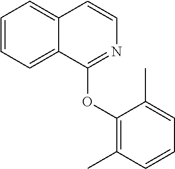 [SMILES: Cc1cccc(C)c1Oc1nccc2ccccc12]

The synthesis method of Example 13 is the same as the general method 1 above.

Reaction yield: 57%; Structural parameters: ¹H NMR (600 MHZ, CDCl₃) δ 8.53 (d, J=7.2 Hz, 1H), 7.93 (d, J=6.0 Hz, 1H), 7.83-7.81 (m, 1H), 7.76-7.74 (m, 1H), 7.67-7.64 (m, 1H), 7.28 (d, J=6.6 Hz, 1H), 7.12-7.15 (m, 2H), 7.13-7.11 (m, 1H), 2.15 (s, 6H).

### Example 14

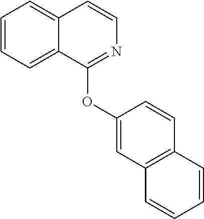 [SMILES: c1ccc2cc(Oc3nccc4ccccc34)ccc2c1]

The synthesis method of Example 14 is the same as the general method 1 above.

Reaction yield: 45%; Structural parameters: ¹H NMR (600 MHZ, CDCl₃) δ 8.64 (d, J=9.6 Hz, 1H), 7.97 (d, J=9.8 Hz, 1H), 7.93-7.91 (m, 2H), 7.87-7.85 (m, 1H), 7.80-7.77 (m, 2H), 7.72-7.69 (m, 1H), 7.57 (t, J=7.8 Hz, 1H), 7.52-7.49 (m, 1H), 7.44-7.40 (m, 2H), 7.34 (d, J=6.6 Hz, 1H).

### Example 15

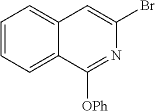 [SMILES: Brc1cc2ccccc2c(Oc2ccccc2)n1]

The synthesis method of Example 15 is the same as the general method 1 above.

Reaction yield: 76%; Structural parameters: ¹H NMR (600 MHZ, CDCl₃) δ 8.44 (d, J=8.4 Hz, 1H), 8.13-8.10 (m, 2H), 7.85-7.82 (m, 1H), 7.69-7.67 (m, 1H), 7.46-7.43 (m, 2H), 7.27-7.23 (m, 3H).

### Example 16

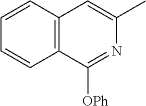 [SMILES: Cc1cc2ccccc2c(Oc2ccccc2)n1]

The synthesis method of Example 16 is the same as the general method 1 above.

Reaction yield: 43%; Structural parameters: ¹H NMR (600 MHZ, CDCl₃) δ 8.37 (d, J=8.4 Hz, 1H), 7.72-7.66 (m, 2H), 7.55-7.52 (m, 1H), 7.45-7.42 (m, 2H), 7.30-7.28 (m, 2H), 7.24-7.21 (m, 1H), 7.18 (s, 1H), 2.48 (s, 3H).

### Example 17

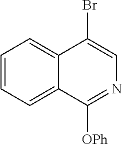 [SMILES: Brc1cnc(Oc2ccccc2)c2ccccc12]

The synthesis method of Example 17 is the same as the general method 1 above.

Reaction yield: 53%; Structural parameters: ¹H NMR (600 MHZ, CDCl₃) δ 8.46 (d, J=8.4 Hz, 1H), 8.15-8.12 (m, 2H), 7.86-7.84 (m, 1H), 7.71-7.68 (m, 1H), 7.47-7.44 (m, 2H), 7.29-7.26 (m, 1H), 7.25-7.24 (m, 2H).

### Example 18

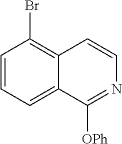 [SMILES: Brc1cccc2c(Oc3ccccc3)nccc12]

The synthesis method of Example 18 is the same as the general method 1 above.

Reaction yield: 62%; Structural parameters: ¹H NMR (600 MHZ, CDCl₃) δ 8.43 (d, J=8.4 Hz, 1H), 8.05 (d, J=6.0 Hz, 1H), 7.99-7.98 (m, 1H), 7.64-7.63 (m, 1H), 7.47-7.43 (m, 3H), 7.27-7.23 (m, 3H).

### Example 19

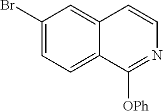 [SMILES: Brc1ccc2c(Oc3ccccc3)nccc2c1]

The synthesis method of Example 19 is the same as the general method 1 above.

Reaction yield: 33%; Structural parameters: ¹H NMR (600 MHZ, CDCl₃) δ 8.31 (d, J=9.0 Hz, 1H), 7.99-7.98 (m, 2H), 7.72-7.70 (m, 1H), 7.46 (t, J=7.8 Hz, 2H), 7.29-7.27 (m, 1H), 7.26-7.22 (m, 3H).

### Example 20

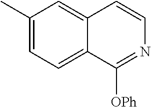 [SMILES: Cc1ccc2c(Oc3ccccc3)nccc2c1]

The synthesis method of Example 20 is the same as the general method 1 above.

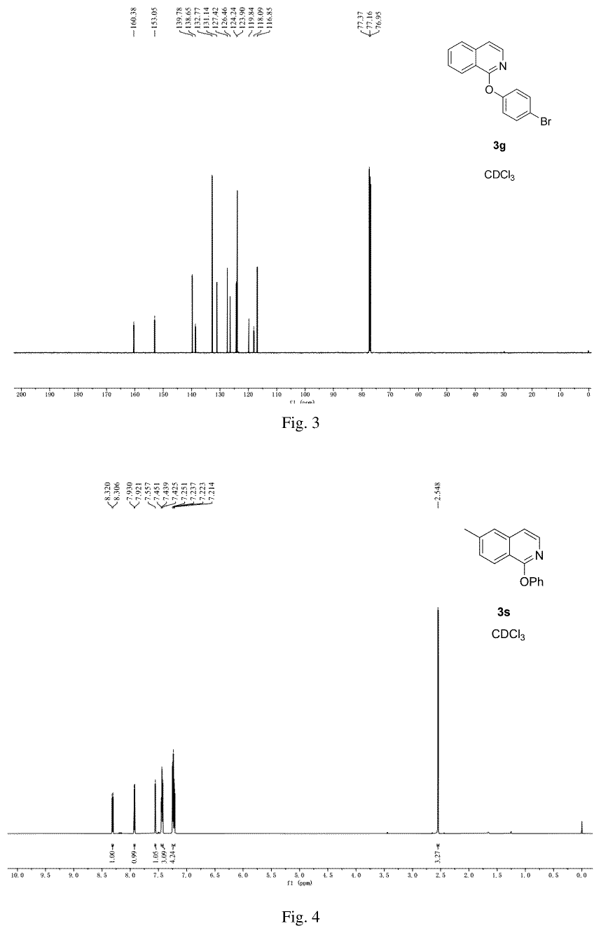

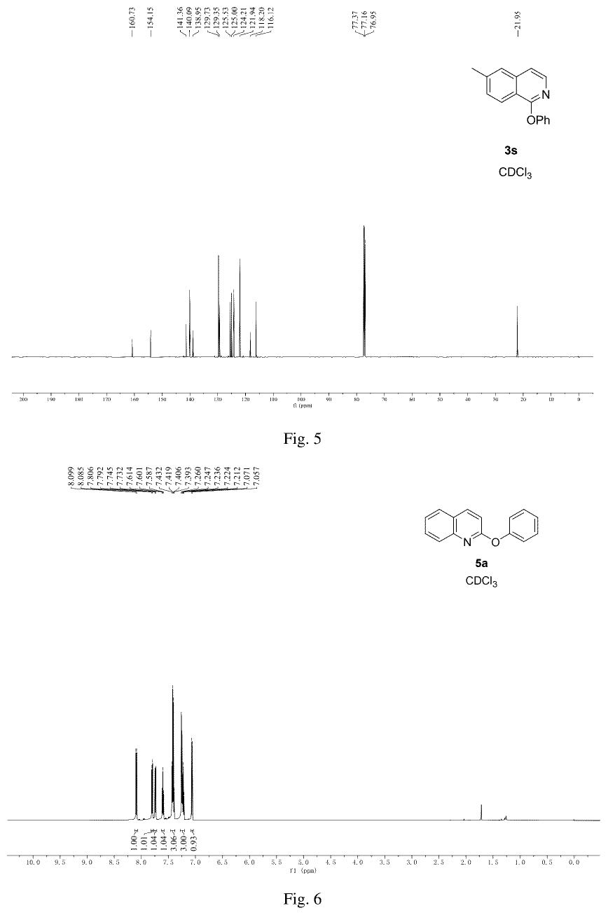

Reaction yield: 65%; Structural parameters: ¹H NMR (600 MHz, CDCl₃) δ 8.31 (d, J=8.4 Hz, 1H), 7.93 (d, J=6.0 Hz, 1H), 7.56 (s, 1H), 7.45-7.43 (m, 3H), 7.25-7.21 (m, 4H), 2.55 (s, 3H). The ¹H NMR spectrum is shown in FIG. 4, and the 13C NMR spectrum is shown in FIG. 5.

### Example 21

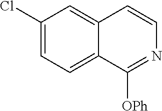 [SMILES: Clc1ccc2c(Oc3ccccc3)nccc2c1]

The synthesis method of Example 21 is the same as the general method 1 above.

Reaction yield: 50%; Structural parameters: ¹H NMR (600 MHZ, CDCl₃) δ 8.37 (d, J=9.0 Hz, 1H), 7.97 (d, J=6.0 Hz, 1H), 7.78 (d, J=1.8 Hz, 1H), 7.55 (dd, J=9.0 Hz, 2.4 Hz, 1H), 7.47-7.44 (m, 2H), 7.27-7.21 (m, 4H).

### Example 22

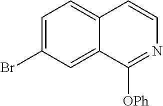 [SMILES: Brc1ccc2ccnc(Oc3ccccc3)c2c1]

The synthesis method of Example 22 is the same as the general method 1 above.

Reaction yield: 50%; Structural parameters: ¹H NMR (600 MHZ, CDCl₃) δ 8.61 (s, 1H), 7.98 (d, J=6.0 Hz, 1H), 7.80-7.79 (m, 1H), 7.68-7.66 (m, 1H), 7.47-7.44 (m, 2H), 7.28-7.27 (m, 2H), 7.25-7.24 (m, 2H).

### Example 23

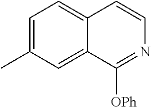 [SMILES: Cc1ccc2ccnc(Oc3ccccc3)c2c1]

The synthesis method of Example 23 is the same as the general method 1 above.

Reaction yield: 38%; Structural parameters: ¹H NMR (600 MHZ, CDCl₃) δ 8.22 (s, 1H), 7.91 (d, J=6.0 Hz, 1H), 7.71-7.70 (m, 1H), 7.57-7.55 (m, 1H), 7.46-7.44 (m, 2H), 7.28 (d, J=6.0 Hz, 1H), 7.26-7.24 (m, 3H), 2.58 (s, 3H).

### Example 24

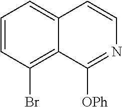 [SMILES: Brc1cccc2ccnc(Oc3ccccc3)c12]

The synthesis method of Example 24 is the same as the general method 1 above.

Reaction yield: 54%; Structural parameters: ¹H NMR (600 MHZ, CDCl₃) δ 7.97 (d, J=6.0 Hz, 1H), 7.93-7.92 (m, 1H), 7.74-7.72 (m, 1H), 7.47-7.44 (m, 3H), 7.32 (d, J=5.4 Hz, 1H), 7.26-7.23 (m, 3H).

### Example 25

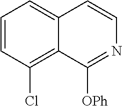 [SMILES: Clc1cccc2ccnc(Oc3ccccc3)c12]

The synthesis method of Example 25 is the same as the general method 1 mentioned above.

Reaction yield: 73%; Structural parameters: ¹H NMR (600 MHz, CDCl₃) δ 7.97 (d, J=5.4 Hz, 1H), 7.69-7.66 (m, 2H), 7.57-7.54 (m, 1H), 7.47-7.44 (m, 2H), 7.31 (d, J=5.4 Hz, 1H), 7.26-7.24 (m, 3H).

### Example 26

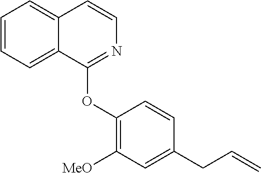 [SMILES: C=CCc1ccc(Oc2nccc3ccccc23)c(OC)c1]

The synthesis method of Example 26 is the same as the general method 1 above.

Reaction yield: 47%; Structural parameters: ¹H NMR (600 MHz, CDCl₃) δ 8.49 (d, J=8.4 Hz, 1H), 7.95 (d, J=6.0 Hz, 1H), 7.80-7.78 (m, 1H), 7.73-7.70 (m, 1H), 7.63-7.60 (m, 1H), 7.28 (d, J=6.0 Hz, 1H), 7.16 (d, J=7.8 Hz, 1H), 6.89-6.86 (m, 2H), 6.06-6.00 (m, 1H), 5.17-5.11 (m, 2H), 3.73 (s, 3H), 3.44 (d, J=6.6 Hz, 2H).

### Example 27

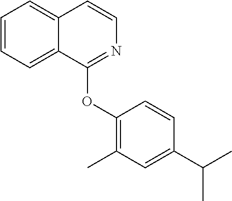 [SMILES: Cc1cc(C(C)C)ccc1Oc1nccc2ccccc12]

The synthesis method of Example 27 is the same as the general method 1 above.

Reaction yield: 65%; Structural parameters: ¹H NMR (600 MHZ, CDCl₃) δ 8.49 (d, J=9.0 Hz, 1H), 7.97 (d, J=6.0 Hz, 1H), 7.82-7.81 (m, 1H), 7.75-7.73 (m, 1H), 7.66-7.63 (m, 1H), 7.29 (d, J=7.2 Hz, 1H), 7.25-7.24 (m, 1H), 7.09-7.08 (m, 1H), 7.06-7.05 (m, 1H), 2.97-2.90 (m, 1H), 2.15 (s, 3H), 1.28 (d, J=7.2 Hz, 6H).

### Example 28

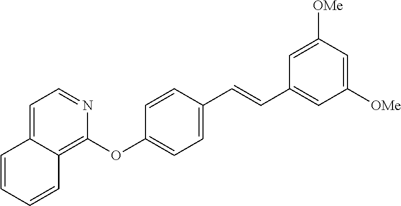 [SMILES: COc1cc(/C=C/c2ccc(Oc3nccc4ccccc34)cc2)cc(OC)c1]

The synthesis method of Example 28 is the same as the general method 1 above.

Reaction yield: 44%; Structural parameters: ¹H NMR (600 MHZ, CDCl₃) δ 8.46 (d, J=8.4 Hz, 1H), 8.01 (d, J=6.0 Hz, 1H), 7.83-7.81 (m, 1H), 7.76-7.73 (m, 1H), 7.66-7.63 (m, 1H), 7.61 (d, J=8.4 Hz, 2H), 7.34 (d, J=6.0 Hz, 1H), 7.30-7.28 (m, 2H).), 7.16-7.13 (m, 1H), 7.05-7.02 (m, 1H), 6.71 (d, J=1.8 Hz, 2H), 6.43 (t, J=2.4 Hz, 1H), 3.86 (s, 6H).

### Example 29

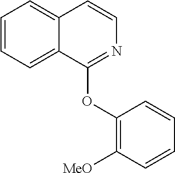 [SMILES: COc1ccccc1Oc1nccc2ccccc12]

The synthesis method of Example 29 is the same as the general method 1 above.

Reaction yield: 40%; Structural parameters: ¹H NMR (600 MHZ, CDCl₃) δ 8.50 (d, J=8.4 Hz, 1H), 7.94 (d, J=6.0 Hz, 1H), 7.79-7.78 (m, 1H), 7.72-7.69 (m, 1H), 7.63-7.60 (m, 1H), 7.28 (d, J=6.0 Hz, 1H), 7.26-7.23 (m, 2H), 7.06-7.03 (m, 2H), 3.73 (s, 3H).

Method 2: A green synthesis method for 2-aryloxyquinoline, the steps are as follows: To a solution of an quinoline N-oxide derivative (1.0 mmol) in dry EA (1.0 mL) is added ArOH (1.5 mmol), DIEA (2.0 mmol) and HMPA (0.1 mmol). The resulting solution is cooled to 0° C., followed by the dropwise addition of a solution of POBr₃ (1.0 mmol) in EA (1.0 mL). The reaction mixture is then warmed to room temperature and stirred for 5 minutes. The reaction is quenched with a saturated potassium carbonate solution and the organic phase is separated. The aqueous phase is extracted once with EA. The organic phase is combined, dried over Na₂SO₄ and concentrated in vacuo to give the crude product. Purification by flash column chromatography (PE:EA=200:1-100:1) furnishes the desired product.

The present invention is further illustrated in detail by the following Examples.

### Example 30

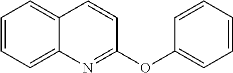 [SMILES: c1ccc(Oc2ccc3ccccc3n2)cc1]

To a solution of an quinoline N-oxide derivative (100 mg, 0.69 mmol) in dry EA (0.7 mL) was added phenol (98 mg, 1.0 mmol), DIEA (181 mg, 1.4 mmol) and HMPA (13 mg, 0.07 mmol). The resulting solution was cooled to 0° C., followed by the dropwise addition of a solution of POBr₃ (198 mg, 0.69 mmol) in EA (0.7 mL). The reaction mixture was then warmed to room temperature and stirred for 5 minutes. The reaction was quenched with a saturated potassium carbonate solution and the organic phase was separated. The aqueous phase was extracted once with EA. The organic phase was combined, dried over Na₂SO₄ and concentrated in vacuo to give the crude product. Purification by flash column chromatography (PE:EA=200:1-100:1) furnished the desired product (106 mg, 69% yield). Structural parameters: ¹H NMR (600 MHZ, CDCl₃) δ 8.09 (d, J=8.4 Hz, 1H), 7.80 (d, J=8.4 Hz, 1H), 7.74 (d, J=7.8 Hz, 1H), 7.60 (t, J=7.8 Hz, 1H), 7.43-7.39 (m, 3H), 7.26-7.21 (m, 3H), 7.06 (d, J=8.4 Hz, 1H). ¹H NMR spectrum is shown in FIG. 6.

### Example 31

To a solution of an quinoline N-oxide derivative (1.0 g, 6.9 mmol) in dry EA (7 mL) was added phenol (0.98 g, 10.4 mmol), DIEA (1.78 g, 13.8 mmol) and HMPA (0.12 g, 0.69 mmol). The resulting solution was cooled to 0° C., followed by the dropwise addition of a solution of POBr₃ (1.98 g, 6.9 mmol) in EA (7 mL). The reaction mixture was then warmed to room temperature and stirred for 5 minutes. The reaction was quenched with a saturated potassium carbonate solution and the organic phase was separated. The aqueous phase was extracted once with EA. The organic phase was combined, dried over Na₂SO₄ and concentrated in vacuo to give the crude product. Purification by flash column chromatography (PE:EA=200:1-100:1) furnished the desired product (950 mg, 62%). Structural parameters: ¹H NMR (600 MHZ, CDCl₃) & 8.09 (d, J=8.4 Hz, 1H), 7.80 (d, J=8.4 Hz, 1H), 7.74 (d, J=7.8 Hz, 1H), 7.60 (t, J=7.8 Hz, 1H), 7.43-7.39 (m, 3H), 7.26-7.21 (m, 3H), 7.06 (d, J=8.4 Hz, 1H). ¹H NMR spectrum is shown in FIG. 6.

### Example 32

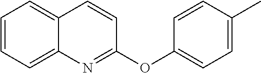 [SMILES: Cc1ccc(Oc2ccc3ccccc3n2)cc1]

The synthesis method of Example 32 is the same as the general method 2 above.

Reaction yield: 87%; Structural parameters: ¹H NMR (600 MHZ, CDCl₃) δ 8.10 (d, J=9.0 Hz, 1H), 7.82 (d, J=8.4 Hz, 1H), 7.75 (d, J=8.4 Hz, 1H), 7.63-7.60 (m, 1H), 7.42 (t, J=7.2 Hz, 1H), 7.24-7.23 (m, 2H), 7.16-7.15 (m, 2H), 7.07 (d, J=9.0). Hz, 1H), 2.40 (s, 3H).

### Example 33

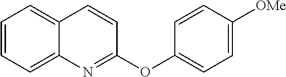 [SMILES: COc1ccc(Oc2ccc3ccccc3n2)cc1]

The synthesis method of Example 33 is the same as the general method 2 above.

Reaction yield: 79%; Structural parameters: ¹H NMR (600 MHZ, CDCl₃) δ 8.09 (d, J=9.0 Hz, 1H), 7.80 (d, J=8.4 Hz, 1H), 7.75 (d, J=7.8 Hz, 1H), 7.61 (t, J=7.2 Hz, 1H), 7.41 (t, J=7.2 Hz, 1H), 7.20-7.18 (m, 2H), 7.05 (d, J=9.0 Hz, 1H), 6.97-6.95 (m, 2H), 3.84 (s, 3H).

### Example 34

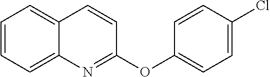 [SMILES: Clc1ccc(Oc2ccc3ccccc3n2)cc1]

The synthesis method of Example 34 is the same as the general method 2 above.

Reaction yield: 63%; Structural parameters: ¹H NMR (600 MHZ, CDCl₃) δ 8.13 (d, J=9.0 Hz, 1H), 7.79-7.76 (m, 2H), 7.64-7.61 (m, 1H), 7.44 (t, J=7.8 Hz, 1H), 7.40-7.38 (m, 2H), 7.23-7.21 (m, 2H), 7.09 (d, J=9.0 Hz, 1H).

### Example 35

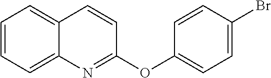 [SMILES: Brc1ccc(Oc2ccc3ccccc3n2)cc1]

The synthesis method of Example 35 is the same as the general method 2 above.

Reaction yield: 55%; Structural parameters: ¹H NMR (600 MHZ, CDCl₃) δ 8.14 (d, J=9.0 Hz, 1H), 7.78 (t, J=9.0 Hz, 2H), 7.64-7.61 (m, 1H), 7.55-7.52 (m, 2H), 7.44 (t, J=7.2 Hz, 1H), 7.18-7.15 (m, 2H), 7.09 (d, J=9.0 Hz, 1H).

### Example 36

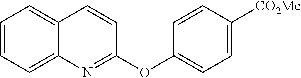 [SMILES: COC(=O)c1ccc(Oc2ccc3ccccc3n2)cc1]

The synthesis method of Example 36 is the same as the general method 2 above.

Reaction yield: 52%; Structural parameters: ¹H NMR (600 MHZ, CDCl₃) δ 8.16 (d, J=9.0 Hz, 1H), 8.12-8.11 (m, 2H), 7.79 (t, J=7.8 Hz, 2H), 7.64 (t, J=8.4 Hz, 1H), 7.46 (t, J=7.2 Hz, 1H), 7.33-7.32 (m, 2H), 7.13 (d, J=9.0 Hz, 1H), 3.93 (s, 3H).

### Example 37

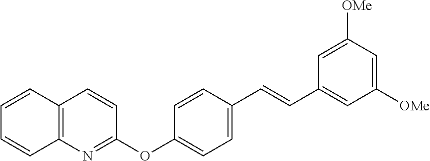 [SMILES: COc1cc(/C=C/c2ccc(Oc3ccc4ccccc4n3)cc2)cc(OC)c1]

The synthesis method of Example 37 is the same as the general method 2 above.

Reaction yield: 68%; Structural parameters: ¹H NMR (600 MHZ, CDCl₃) δ 8.13 (d, J=9.0 Hz, 1H), 7.81 (d, J=8.4 Hz, 1H), 7.76 (d, J=8.4 Hz, 1H), 7.62 (t, J=8.4 Hz, 1H), 7.56 (d, J=9.0 Hz, 2H), 7.43 (t, J=7.2 Hz, 1H), 7.26-7.25 (m, 2H), 7.13-7.09 (m, 2H), 7.03-7.00 (m, 1H), 6.68 (d, J=2.4 Hz, 2H), 6.41-6.40 (m, 1H), 3.84 (s, 6H).

### Example 38

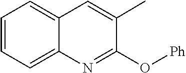 [SMILES: Cc1cc2ccccc2nc1Oc1ccccc1]

The synthesis method of Example 38 is the same as the general method II above.

Reaction yield: 41%; Structural parameters: ¹H NMR (600 MHZ, CDCl₃) δ 7.91 (s, 1H), 7.70 (dd, J=16.2 Hz, 8.4 Hz, 2H), 7.52-7.50 (m, 1H), 7.43-7.41 (m, 2H), 7.38-7.36 (m, 1H), 7.26-7.25 (m, 2H), 7.23-7.20 (m, 1H), 2.50 (s, 3H).

### Example 39

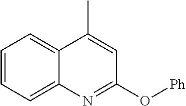 [SMILES: Cc1cc(Oc2ccccc2)nc2ccccc12]

The synthesis method of Example 39 is the same as the general method 2 mentioned above.

Reaction yield: 54%; Structural parameters: ¹H NMR (600 MHz, CDCl₃) δ 7.90 (d, J=8.4 Hz, 1H), 7.81 (d, J=8.4 Hz, 1H), 7.61-7.59 (m, 1H), 7.44-7.40 (m, 3H), 7.24-7.23 (m, 2H), 7.22-7.20 (m, 1H), 6.91 (s, 1H), 2.66 (s, 3H).

### Example 40

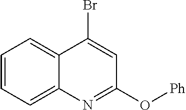 [SMILES: Brc1cc(Oc2ccccc2)nc2ccccc12]

The synthesis method of Example 40 is the same as the general method 2 above.

Reaction yield: 63%; Structural parameters: ¹H NMR (600 MHZ, CDCl₃) δ 8.10 (d, J=9.6 Hz, 1H), 7.78-7.77 (m, 1H), 7.65-7.63 (m, 1H), 7.51-7.48 (m, 1H), 7.44-7.42 (m, 2H), 7.40 (s, 1H), 7.25-7.23 (m, 3H).

### Example 41

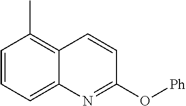 [SMILES: Cc1cccc2nc(Oc3ccccc3)ccc12]

The synthesis method of Example 41 is the same as the above general method two.

Reaction yield: 54%; Structural parameters: ¹H NMR (600 MHZ, CDCl₃) δ 8.29 (d, J=9.0 Hz, 1H), 7.66 (d, J=8.4 Hz, 1H), 7.50 (t, J=7.2 Hz, 1H), 7.42 (t, J=7.8 Hz, 2H), 7.26-7.22 (m, 4H), 7.08 (d, J=9.0 Hz, 1H), 2.66 (s, 3H).

### Example 42

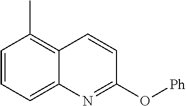 [SMILES: Cc1cccc2nc(Oc3ccccc3)ccc12]

The synthesis method of Example 42 is the same as the general method 2 above.

Reaction yield: 57%; Structural parameters: ¹H NMR (600 MHZ, CDCl₃) δ 8.04 (d, J=8.4 Hz, 1H), 7.72 (d, J=8.4 Hz, 1H), 7.53 (s, 1H), 7.46 (dd, J=9.0 Hz, 2.4 Hz, 1H), 7.43-7.41 (m, 2H), 7.26-7.21 (m, 3H), 7.04 (d, J=9.0 Hz, 1H), 2.50 (s, 3H).

### Example 43

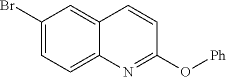 [SMILES: Brc1ccc2nc(Oc3ccccc3)ccc2c1]

The synthesis method of Example 43 is the same as the general method 2 above.

Reaction yield: 71%; Structural parameters: ¹H NMR (600 MHZ, CDCl₃) δ 8.03 (d, J=9.0 Hz, 1H), 7.91 (d, J=1.8 Hz, 1H), 7.69-7.65 (m, 2H), 7.44 (t, J=8.4 Hz, 2H), 7.27-7.24 (m, 3H), 7.11 (d, J=9.0 Hz, 1H).

### Example 44

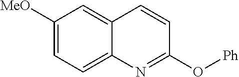 [SMILES: COc1ccc2nc(Oc3ccccc3)ccc2c1]

The synthesis method of Example 44 is the same as the above general method 2 above.

Reaction yield: 58%; Structural parameters: ¹H NMR (600 MHZ, CDCl₃) δ 8.03 (d, J=9.0 Hz, 1H), 7.73 (d, J=9.0 Hz, 1H), 7.41 (t, J=8.4 Hz, 2H), 7.29 (dd, J=3.0 Hz, 9.6 Hz, 1H), 7.24-7.20 (m, 3H), 7.08 (d, J=3.0 Hz, 1H), 7.05 (d, J=9.0 Hz, 1H), 3.91 (s, 3H).

### Example 45

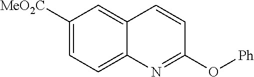 [SMILES: COC(=O)c1ccc2nc(Oc3ccccc3)ccc2c1]

The synthesis method of Example 45 is the same as the general method 2 mentioned above.

Reaction yield: 62%; Structural parameters: ¹H NMR (600 MHZ, CDCl₃) δ 8.51 (d, J=1.8 Hz, 1H), 8.21-8.19 (m, 2H), 7.79 (d, J=8.4 Hz, 1H), 7.45 (t, J=7.8 Hz, 2H), 7.27-7.26 (m, 3H), 7.14 (d, J=8.4 Hz, 1H), 3.97 (s, 3H).

### Example 46

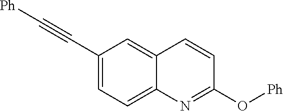 [SMILES: C(#Cc1ccc2nc(Oc3ccccc3)ccc2c1)c1ccccc1]

The synthesis method of Example 46 is the same as the general method 2 above.

Reaction yield: 42%; Structural parameters: ¹H NMR (600 MHZ, CDCl₃) δ 8.10 (d, J=9.0 Hz, 1H), 7.98 (s, 1H), 7.78-7.73 (m, 2H), 7.59-7.56 (m, 2H), 7.46 (t, J=7.8 Hz, 2H), 7.40-7.36 (m, 3H), 7.29-7.27 (m, 3H), 7.12 (d, J=9.0 Hz, 1H).

### Example 47

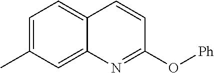 [SMILES: Cc1ccc2ccc(Oc3ccccc3)nc2c1]

The synthesis method of Example 47 is the same as the general method 2 mentioned above.

Reaction yield: 40%; Structural parameters: ¹H NMR (600 MHZ, CDCl₃) δ 8.07 (d, J=9.0 Hz, 1H), 7.65 (d, J=8.4 Hz, 1H), 7.61 (s, 1H), 7.44-7.41 (m, 2H), 7.27-7.22 (m, 4H), 7.01 (d, J=9.0 Hz, 1H), 2.49 (s, 3H).

### Example 48

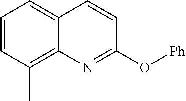 [SMILES: Cc1cccc2ccc(Oc3ccccc3)nc12]

The synthesis method of Example 48 is the same as the general method 2 above.

Reaction yield: 30%; Structural parameters: ¹H NMR (600 MHZ, CDCl₃) δ 8.09 (d, J=8.4 Hz, 1H), 7.60 (d, J=7.8 Hz, 1H), 7.48 (d, J=6.6 Hz, 1H), 7.44-7.41 (m, 2H), 7.33-7.31 (m, 3H), 7.23 (t, J=7.8 Hz, 1H), 7.06 (d, J=8.4 Hz, 1H), 2.55 (s, 3H).

Method 3: A green synthesis method for 2-aryloxypyridine, the steps are as follows: To a solution of a pyridine N-oxide derivative (1.0 mmol) in dry EA (1.0 mL) is added ArOH (2.0 mmol), DIEA (2.0 mmol) and HMPA (0.2 mmol). The resulting solution is cooled to 0° C., followed by the dropwise addition of a solution of POBr₃ (2.0 mmol) in EA (1.0 mL). The reaction mixture is then warmed to room temperature and stirred for 5 minutes. The reaction is quenched with a saturated potassium carbonate solution and the organic phase is separated. The aqueous phase is extracted once with EA. The organic phase is combined, dried over Na₂SO₄ and concentrated in vacuo to give the crude product. Purification by flash column chromatography (PE:EA=200:1-100:1) furnishes the desired product.

The present invention is further illustrated in detail by the following Examples.

### Example 49

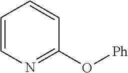 [SMILES: c1ccc(Oc2ccccn2)cc1]

To a solution of a pyridine N-oxide derivative (100 mg, 1.05 mmol) in EA (1.1 mL) was added phenol (198 mg, 2.1 mmol), DIEA (271 mg, 2.1 mmol) and HMPA ((38 mg, 0.21 mmol). The resulting solution was cooled to 0° C., followed by the dropwise addition of a solution of POBr₃ (602 mg, 2.1 mmol) in EA (1.1 mL). The reaction mixture was then warmed to room temperature and stirred for 5 minutes. The reaction was quenched with a saturated potassium carbonate solution and the organic phase was separated. The aqueous phase was extracted once with EA. The organic phase was combined, dried over Na₂SO₄ and concentrated in vacuo to give the crude product. Purification by flash column chromatography (PE:EA=200:1-100:1) furnished the desired product (104 mg, 58% yield). Structural parameters: ¹H NMR (600 MHZ, CDCl₃) δ 8.22-8.21 (m, 1H), 7.69-7.67 (m, 1H), 7.44-7.39 (m, 2H), 7.22-7.19 (m, 1H), 7.16-7.14 (m, 2H), 7.00-6.98 (m, 1H), 6.91 (d, J=8.4 Hz, 1H).

### Example 50

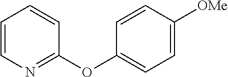 [SMILES: COc1ccc(Oc2ccccn2)cc1]

The synthesis method of Example 50 is the same as the general method 3 above.

Reaction yield: 47%; Structural parameters: ¹H NMR (600 MHZ, CDCl₃) δ 8.19-8.18 (m, 1H), 7.67-7.64 (m, 1H), 7.08-7.07 (m, 2H), 6.96-6.92 (m, 3H), 6.86 (d, J=8.4 Hz, 1H), 3.81 (s, 3H).

### Example 51

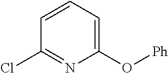 [SMILES: Clc1cccc(Oc2ccccc2)n1]

The synthesis method of Example 51 is the same as the general method 3 above.

Reaction yield: 42%; Structural parameters: ¹H NMR (600 MHz, CDCl₃) δ 7.61 (t, J=7.8 Hz, 1H), 7.42-7.39 (m, 2H), 7.22 (t, J=7.2 Hz, 1H), 7.15-7.14 (m, 2H), 7.03 (d, J=7.8 Hz, 1H), 6.74 (d, J=7.8 Hz, 1H).

### Example 52

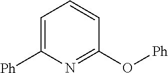 [SMILES: c1ccc(Oc2cccc(-c3ccccc3)n2)cc1]

The synthesis method of Example 52 is the same as the general method 3 above.

Reaction yield: 63%; Structural parameters: ¹H NMR (600 MHz, CDCl₃) δ 7.79-7.78 (m, 2H), 7.52 (t, J=7.8 Hz, 1H), 7.29 (d J=7.8 Hz, 1H), 7.27-7.23 (m, 4H), 7.21-7.18 (m, 1H), 7.09-7.04 (m, 3H), 6.61 (d, J=7.8 Hz, 1H).

### Example 53

The synthesis method of Example 53 is the same as the general method 3 above, and two isomeric products are obtained:

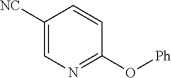 [SMILES: N#Cc1ccc(Oc2ccccc2)nc1]

7e-1: Reaction yield: 39%; Structural parameters: ¹H NMR (600 MHZ, CDCl₃) & 8.47 (d, J=2.4 Hz, 1H), 7.91 (dd, J=8.4 Hz, 2.4 Hz, 1H), 7.46-7.44 (m, 2H), 7.30-7.28 (m, 1H), 7.15-7.14 (m, 2H), 7.02 (d, J=8.4 Hz, 1H).

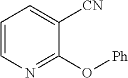 [SMILES: N#Cc1cccnc1Oc1ccccc1]

7e-2: Reaction yield: 26%; Structural parameters: ¹H NMR (600 MHZ, CDCl₃) & 8.31 (dd, J=5.4 Hz, 2.4 Hz, 1H), 8.01 (dd, J=7.2 Hz, 1.8 Hz, 1H), 7.46-7.43 (m, 2H), 7.29-7.27 (m, 1H), 7.19 (d, J=8.4 Hz, 2H), 7.10-7.08 (m, 1H).

### Example 54

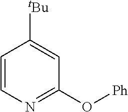 [SMILES: CC(C)(C)c1ccnc(Oc2ccccc2)c1]

The synthesis method of Example 54 is the same as the general method 3 above.

Reaction yield: 44%; Structural parameters: ¹H NMR (600 MHz, CDCl₃) δ 8.10 (d, J=5.4 Hz, 1H), 7.40 (t, J=7.8 Hz, 2H), 7.19 (t, J=7.8 Hz, 1H), 7.15-7.14 (m, 2H), 7.01 (dd, J=5.4 Hz, 1.2 Hz, 1H), 6.93 (t, J=1.8 Hz, 1H), 1.32 (s, 9H).

### Example 55

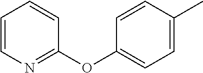

The synthesis method of Example 55 is the same as the general method 3 above.

Reaction yield: 42%; Structural parameters: ¹H NMR (600 MHz, CDCl₃) δ 8.17-8.15 (m, 1H), 7.59 (t, J=6.6 Hz, 1H), 7.16 (d, J=8.1 Hz, 2H), 7.01 (d, J=8.5 Hz, 2H), 6.91-6.89 (m, 1H), 6.84 (d, J=8.3 Hz, 1H), 2.32 (s, 3H).

### Example 56

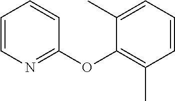

The synthesis method of Example 56 is the same as the general method 3 above.

Reaction yield: 44%; Structural parameters: ¹H NMR (600 MHz, CDCl₃) δ 8.19-8.18 (m, 1H), 7.67-7.64 (m, 1H), 7.14-7.09 (m, 3H), 6.95-6.93 (m, 1H), 6.84 (d, J=8.3 Hz, 1H), 2.16 (s, 6H).

### Example 57

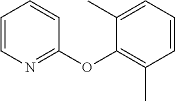

The synthesis method of Example 57 is the same as the general method 3 above.

Reaction yield: 34%; Structural parameters: ¹H NMR (600 MHZ, CDCl₃) δ 8.16 (d, J=4.9 Hz, 1H), 7.73-7.70 (m, 1H), 7.65 (dd, J=8.0, 1.5 Hz, 1H), 7.38-7.35 (m, 1H), 7.21-7.20 (m, 1H), 7.10-7.13 (m, 1H), 7.01-6.97 (m, 2H).

Although the embodiments of the present invention are disclosed for illustrative purposes, a person skilled in the field can understand that all kinds of substitutions, changes and modifications are possible without departing from the spirit and scope of the present invention and the attached claims, and therefore the scope of the present invention is not limited to the content disclosed in the embodiment.

1. A method for synthesizing an aryl ether ortho-substituted to the nitrogen atom of an N-heterocycle, characterized by: reacting an N-heterocyclic N-oxide with a phenol using hexamethylphosphoramide (HMPA) as a catalyst, N,N-diisopropylethylamine (DIEA) as a base, and POBr₃ as an auxiliary agent, thereby effecting ortho-aryloxy substitution relative to the nitrogen atom of an N-heterocycle;
wherein said aryl ether is an aryl heteroaryl ether synthesized according to the following route: 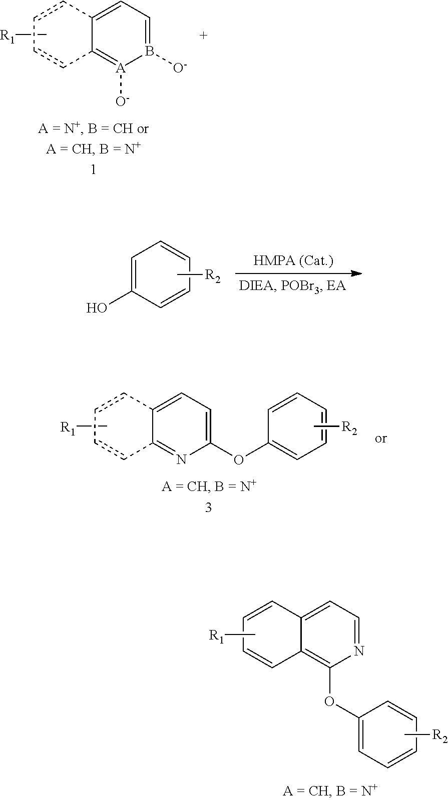
the method comprises reacting a compound of N-heterocyclic N-oxide 1 with a phenol in ethyl acetate (EA) solvent, using N,N-diisopropylethylamine (DIEA) as a base, hexamethylphosphoramide (HMPA) as a catalyst, and POBr₃ as an auxiliary agent, to yield compound 3;
wherein R₁ is selected from the group consisting of C1-C6 alkyl, halogen, C1-C6 ester, phenyl, and phenylethynyl; and R₂ is selected from the group consisting of C1-C6 alkyl, C1-C6 alkenyl, cyano, C1-C6 alkoxy, and halogen.

2. A method for synthesizing an aryl ether ortho-substituted to the nitrogen atom of an N-heterocycle, characterized in that: the aryl ether ortho-substituted to the nitrogen atom of an N-heterocycle is 1-phenoxyisoquinoline, and its synthetic route is as follows: 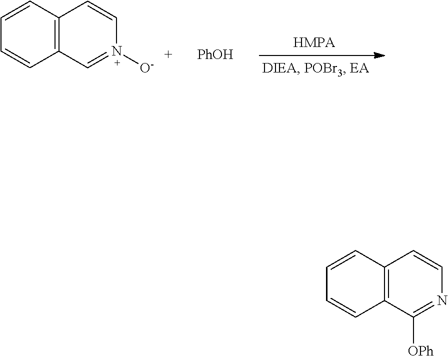
the 1-phenoxyisoquinoline is synthesized by reacting isoquinoline-N-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

3. A method for synthesizing an aryl ether ortho-substituted to the nitrogen atom of an N-heterocycle, characterized in that: the aryl ether ortho-substituted to the nitrogen atom of an N-heterocycle is a pharmaceutical intermediate 6-bromo-1-phenoxyisoquinoline, and its synthetic route is as follows: 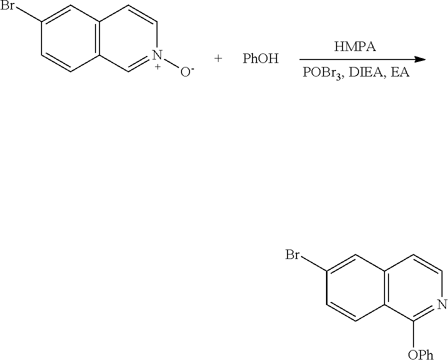
the 6-bromo-1-phenoxyisoquinoline is synthesized by reacting 6-bromoisoquinoline 2-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

4. The method of claim 1, characterized in that: the aryl ether ortho-substituted to the nitrogen atom of an N-heterocycle is a pharmaceutical intermediate 7-bromo-1-phenoxyisoquinoline, and its synthetic route is as follows: 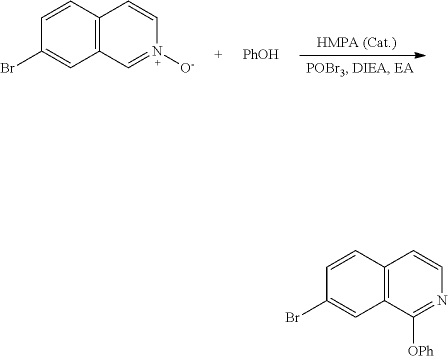
the 7-bromo-1-phenoxyisoquinoline is synthesized by reacting 7-bromoisoquinoline N-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

5. The method of claim 1, characterized in that: the aryl ether ortho-substituted to the nitrogen atom of an N-heterocycle is an intermediate for Factor Xa inhibitors, 7-methyl-1-phenoxyisoquinoline, and its synthetic route is as follows: 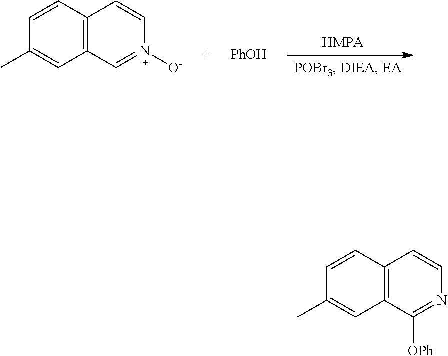
the 7-methyl-1-phenoxyisoquinoline is synthesized by reacting 7-methylisoquinoline 2-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

6. The method of claim 1, characterized in that: the aryl ether ortho-substituted to the nitrogen atom of an N-heterocycle is 2-phenoxyquinoline, and its synthetic route is as follows: 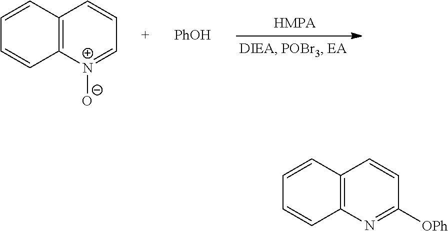 [SMILES: [O-][n+]1cccc2ccccc21 ; "PhOH" ; c1ccc(Oc2ccc3ccccc3n2)cc1]
the 2-phenoxyquinoline is synthesized by reacting quinoline-N-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

7. The method of claim 1, characterized in that: the aryl ether ortho-substituted to the nitrogen atom of an N-heterocycle is a pterostilbene derivative, (E)-2-(4-(3,5-dimethoxystyryl) phenoxy) quinoline, and its synthetic route is as follows: 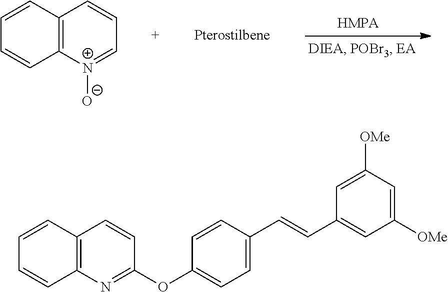 [SMILES: [O-][n+]1cccc2ccccc21 ; "Pterostilbene" ; COc1cc(/C=C/c2ccc(Oc3ccc4ccccc4n3)cc2)cc(OC)c1]
the (E)-2-(4-(3,5-dimethoxystyryl) phenoxy) quinoline is synthesized by reacting quinoline-N-oxide with pterostilbene in ethyl acetate (EA) solvent, using N,N-diisopropylethylamine (DIEA) as a base, hexamethylphosphoramide (HMPA) as a catalyst, and POBr₃ as an auxiliary agent.

8. The method of claim 1, characterized in that: the aryl ether ortho-substituted to the nitrogen atom of an N-heterocycle is an optoelectronic material intermediate, 2-phenoxy-6-phenylpyridine, and its synthetic route is as follows: 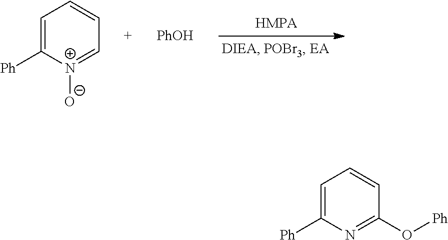 [SMILES: [O-][n+]1ccccc1-c1ccccc1 ; "PhOH" ; c1ccc(Oc2cccc(-c3ccccc3)n2)cc1]
the 2-phenoxy-6-phenylpyridine is synthesized by reacting 2-phenylpyridine 1-oxide with phenol in ethyl acetate (EA) solvent, using N,N-diisopropylethylamine (DIEA) as a base, hexamethylphosphoramide (HMPA) as a catalyst, and POBr₃ as an auxiliary agent.

9. The method of claim 1, characterized in that: the aryl ether ortho-substituted to the nitrogen atom of an N-heterocycle is selected from the group consisting of a 1-aryloxyisoquinoline derivative, a 2-aryloxyquinoline derivative, and a 2-aryloxypyridine derivative;
wherein the 1-aryloxyisoquinoline derivative is selected from: 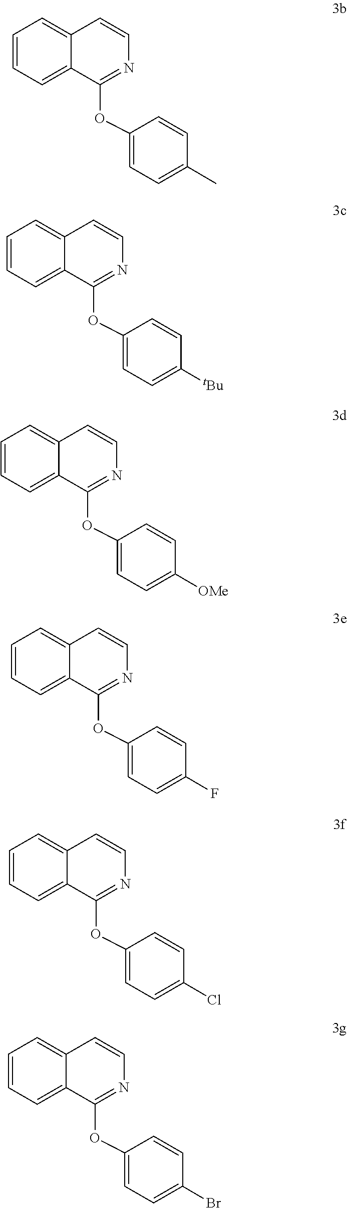 [SMILES: Cc1ccc(Oc2nccc3ccccc23)cc1 ; CC(C)(C)c1ccc(Oc2nccc3ccccc23)cc1 ; COc1ccc(Oc2nccc3ccccc23)cc1 ; Fc1ccc(Oc2nccc3ccccc23)cc1 ; Clc1ccc(Oc2nccc3ccccc23)cc1 ; Brc1ccc(Oc2nccc3ccccc23)cc1] 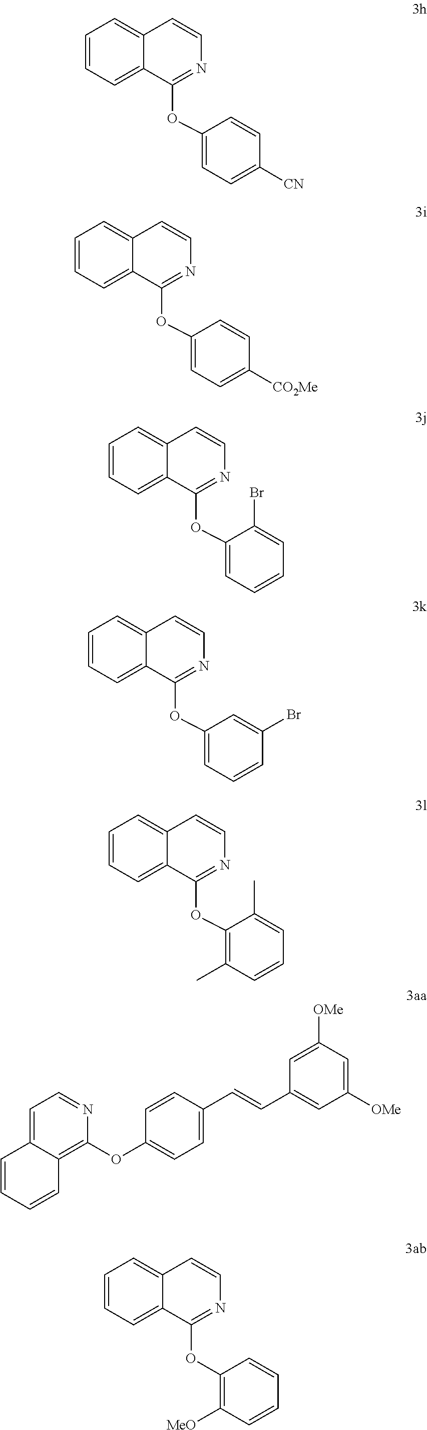
the 2-aryloxyquinoline derivative is selected from: 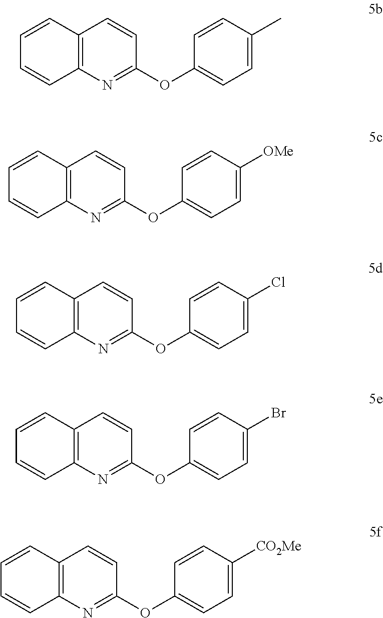 [SMILES: Cc1ccc(Oc2ccc3ccccc3n2)cc1 ; COc1ccc(Oc2ccc3ccccc3n2)cc1 ; Clc1ccc(Oc2ccc3ccccc3n2)cc1 ; Brc1ccc(Oc2ccc3ccccc3n2)cc1 ; COC(=O)c1ccc(Oc2ccc3ccccc3n2)cc1]
and the 2-aryloxypyridine derivative is selected from: [chemical structure]
